(19) United States
(12) Patent Application Publication
(10) Pub. No.: US 2017/0348221 A1
(43) Pub. Date: Dec. 7, 2017

(54) NON-STICKY STABLE COMPOSITION

(71) Applicant: L'OREAL, Paris (FR)

(72) Inventors: Kazuhiko MARUYAMA, Kawasaki-shi, Kanagawa (JP); Yukinori YAMADA, Kawasaki-shi, Kanagawa (JP)

(73) Assignee: L'OREAL, Paris (FR)

(21) Appl. No.: 15/536,193

(22) PCT Filed: Dec. 9, 2015
(86) PCT No.: PCT/JP2015/085128

§ 371 Date: Jun. 15, 2017

(30) Foreign Application Priority Data
Dec. 19, 2014 (JP) 2014-257013

(51) Int. Cl. A61K 8/895; A61K 8/34; A61Q 3/00; A61Q 5/00; A61K 8/37; A61K 8/06; A61K 8/73; A61K 8/39; A61K 8/33; A61Q 19/00

(52) U.S. Cl. CPC A61K 8/895; A61K 8/33; A61K 8/342; A61Q 3/00; A61Q 19/00; A61K 8/37; A61K 8/062; A61K 8/73; A61K 8/39; A61Q 5/00; A61K 2800/548; A61K 2800/594; A61K 2800/10

(57) ABSTRACT

The present invention relates to a composition comprising: (a) at least one oil; (b) at least one polyglyceryl fatty acid ester; (c) at least one silicone elastomer; and (d) at least one polysaccharide. The composition according to the present invention provides no sticky feeling or a reduced sticky feeling to the touch, and is stable, in particular stable over time and/or under elevated temperature, although the composition includes a polyglyceryl fatty acid ester.

### TECHNICAL FIELD

The present invention relates to a composition, preferably a cosmetic or dermatological composition, which is non-sticky and stable.

### BACKGROUND ART

Compositions including polyglyceryl fatty acid esters have been known in the fields of cosmetics and dermatology. Polyglyceryl fatty acid esters can function as surfactants, and therefore, they may be used to prepare, typically, emulsions such as oil-in-water (O/W) or water-in-oil (W/O) emulsions.

For example, JP-A-2007-153858, JP-A-2003-300855 and JP-A-2012-184167 disclose emulsions which are formed by using a polyglyceryl fatty acid ester as a surfactant.

### DISCLOSURE OF INVENTION

However, compositions including a polyglyceryl fatty acid ester often provide a sticky feeling to the touch and tend to be unstable by adding silicones, in particular unstable over time and/or under elevated temperature.

An objective of the present invention is to provide a composition which provides no sticky feeling or a reduced sticky feeling after application and is stable, in particular stable over time and/or under elevated temperature, even if the composition includes a polyglyceryl fatty acid ester and silicones.

The above objective of the present invention can be achieved by a composition comprising:

(a) at least one oil;
(b) at least one polyglyceryl fatty acid ester;
(c) at least one silicone elastomer; and
(d) at least one polysaccharide.

The (a) oil may comprise at least one ester oil, at least one fatty alcohol, or a mixture thereof.

The amount of the (a) oil may range from 0.1 to 35% by weight, preferably from 0.5 to 30% by weight, and more preferably from 1 to 25% by weight, relative to the total weight of the composition.

The (b) polyglyceryl fatty acid ester may have a polyglyceryl moiety derived from 2 to 10 glycerins, preferably 2 to 8 glycerins, and more preferably from 2 to 6 glycerins.

It is preferable that the (b) polyglyceryl fatty acid ester be chosen from polyglyceryl monolaurate comprising 2 to 6 glycerol units, polyglyceryl mono(iso)stearate comprising 2 to 6 glycerol units, polyglyceryl monooleate comprising 2 to 6 glycerol units, polyglyceryl dioleate comprising 2 to 6 glycerol units.

The amount of the (b) polyglyceryl fatty acid ester may range from 0.1 to 20% by weight, preferably from 0.5 to 15% by weight, and more preferably from 1 to 10% by weight, relative to the total weight of the composition.

The weight ratio of the (b) polyglyceryl fatty acid ester to the (a) oil may be 1 or less, preferably 0.8 or less, and more preferably 0.6 or less.

The weight ratio of the (b) polyglyceryl fatty acid ester to the (c) silicone elastomer may be 15 or less, preferably 12 or less, and more preferably 10 or less.

The amount of the (c) silicone elastomer may range from 0.05 to 15% by weight, preferably from 0.1 to 10% by weight, and more preferably from 0.2 to 5% by weight, relative to the total weight of the composition.

It is preferable that the (d) polysaccharide be derived from microorganisms.

The amount of the (d) polysaccharide may range from 0.01 to 10% by weight, preferably from 0.05 to 5% by weight, and more preferably from 0.1 to 1% by weight, relative to the total weight of the composition.

The composition according to the present invention may further comprise water.

The composition according to the present invention may further comprise at least one additional surfactant different from the (b) polyglyceryl fatty acid ester.

The composition according to the present invention may further comprise at least one associative polymer.

The present invention also relates to a cosmetic process for treating the skin, the hair, mucous membranes, the nails, the eyelashes, the eyebrows and/or the scalp, characterized in that the composition according to the present invention is applied to the skin, the hair, mucous membranes, the nails, the eyelashes, the eyebrows or the scalp.

### BEST MODE FOR CARRYING OUT THE INVENTION

After diligent research, the inventors have discovered that it is possible to provide a composition which provides no sticky feeling or a reduced sticky feeling to the touch, even if the composition includes a polyglyceryl fatty acid ester, and is stable, in particular stable over time and/or under elevated temperature, even if the composition includes a silicone, by using a specific combination of selected ingredients.

Thus, one aspect of the present invention is a composition comprising:

(a) at least one oil;
(b) at least one polyglyceryl fatty acid ester;
(c) at least one silicone elastomer; and
(d) at least one polysaccharide.

The composition according to the present invention includes a polyglyceryl fatty acid ester, but can provide no sticky feeling or a reduced sticky feeling to the touch. Therefore, the composition according to the present invention can provide an excellent feel during use, in particular on feeling of the skin after application of the composition.

The term “sticky” here means a property which provides a tacky feeling to the skin.

The composition according to the present invention is stable just after and a long time after the preparation of the composition, even under elevated temperature. Therefore, the composition according to the present invention is stable over time, and can be stored for a long period of time even under hot conditions such as in the summer.

Hereinafter, the composition according to the present invention will be explained in a more detailed manner.

### [Oil]

The composition according to the present invention comprises at least one oil. Here, “oil” means a fatty compound or substance which is in the form of a liquid or a paste (non-solid) at room temperature (25° C.) under atmospheric pressure (760 mmHg). As the oils, those generally used in cosmetics can be used alone or in combination thereof. These oils may be volatile or non-volatile.

The oil may be a non-polar oil such as a hydrocarbon oil, a silicone oil, or the like; a polar oil such as a plant or animal oil and an ester oil or an ether oil; or a mixture thereof

The (a) oil may be selected from the group consisting of oils of plant or animal origin, synthetic oils, silicone oils, hydrocarbon oils and fatty alcohols.

As examples of plant oils, mention may be made of, for example, linseed oil, camellia oil, macadamia nut oil, corn oil, mink oil, olive oil, avocado oil, sasanqua oil, castor oil, safflower oil, jojoba oil, sunflower oil, almond oil, rapeseed oil, sesame oil, soybean oil, peanut oil, and mixtures thereof

As examples of animal oils, mention may be made of, for example, squalene and squalane.

As examples of synthetic oils, mention may be made of alkane oils such as isododecane and isohexadecane, ester oils, ether oils, and artificial triglycerides.

The ester oils are preferably liquid esters of saturated or unsaturated, linear or branched C₁-C₂₆ aliphatic monoacids or polyacids and of saturated or unsaturated, linear or branched C₁-C₂₆ aliphatic monoalcohols or polyalcohols, the total number of carbon atoms of the esters being greater than or equal to 10.

Preferably, for the esters of monoalcohols, at least one from among the alcohol and the acid from which the esters of the present invention are derived is branched.

Among the monoesters of monoacids and of monoalcohols, mention may be made of ethyl palmitate, ethyl hexyl palmitate, isopropyl palmitate, dicaprylyl carbonate, alkyl myristates such as isopropyl myristate or ethyl myristate, isocetyl stearate, 2-ethylhexyl isononanoate, isononyl isononanoate, isodecyl neopentanoate and isostearyl neopentanoate.

Esters of C₄-C₂₂ dicarboxylic or tricarboxylic acids and of C₁-C₂₂ alcohols, and esters of monocarboxylic, dicarboxylic or tricarboxylic acids and of non-sugar C₄-C₂₆ dihydroxy, trihydroxy, tetrahydroxy or pentahydroxy alcohols may also be used.

Mention may especially be made of: diethyl sebacate; isopropyl lauroyl sarcosinate; diisopropyl sebacate; bis(2-ethylhexyl) sebacate; diisopropyl adipate; di-n-propyl adipate; dioctyl adipate; bis(2-ethylhexyl) adipate; diisostearyl adipate; bis(2-ethylhexyl) maleate; triisopropyl citrate; triisocetyl citrate; triisostearyl citrate; glyceryl trilactate; glyceryl trioctanoate; trioctyldodecyl citrate; trioleyl citrate; neopentyl glycol diheptanoate; diethylene glycol diisononanoate.

As ester oils, one can use sugar esters and diesters of C₆-C₃₀ and preferably C₁₂-C₂₂ fatty acids. It is recalled that the term “sugar” means oxygen-bearing hydrocarbon-based compounds containing several alcohol functions, with or without aldehyde or ketone functions, and which comprise at least 4 carbon atoms. These sugars may be monosaccharides, oligosaccharides or polysaccharides.

Examples of suitable sugars that may be mentioned include sucrose (or saccharose), glucose, galactose, ribose, fucose, maltose, fructose, mannose, arabinose, xylose and lactose, and derivatives thereof, especially alkyl derivatives, such as methyl derivatives, for instance methylglucose.

The sugar esters of fatty acids may be chosen especially from the group comprising the esters or mixtures of esters of sugars described previously and of linear or branched, saturated or unsaturated C₆-C₃₀ and preferably C₁₂-C₂₂ fatty acids. If they are unsaturated, these compounds may have one to three conjugated or non-conjugated carbon-carbon double bonds.

The esters according to this variant may also be selected from monoesters, diesters, triesters, tetraesters and polyesters, and mixtures thereof.

These esters may be, for example, oleates, laurates, palmitates, myristates, behenates, cocoates, stearates, linoleates, linolenates, caprates and arachidonates, or mixtures thereof such as, especially, oleopalmitate, oleostearate and palmitostearate mixed esters, as well as pentaerythrityl tetraethyl hexanoate.

More particularly, use is made of monoesters and diesters and especially sucrose, glucose or methylglucose monooleates or dioleates, stearates, behenates, oleopalmitates, linoleates, linolenates and oleostearates.

An example that may be mentioned is the product sold under the name Glucate® DO by the company Amerchol, which is a methylglucose dioleate.

As examples of preferable ester oils, mention may be made of, for example, diisopropyl adipate, dioctyl adipate, 2-ethylhexyl hexanoate, ethyl laurate, cetyl octanoate, octyldodecyl octanoate, isodecyl neopentanoate, myristyl propionate, 2-ethylhexyl 2-ethylhexanoate, 2-ethylhexyl octanoate, 2-ethylhexyl caprylate/caprate, methyl palmitate, ethyl palmitate, isopropyl palmitate, dicaprylyl carbonate, isopropyl lauroyl sarcosinate, isononyl isononanoate, ethylhexyl palmitate, isohexyl laurate, hexyl laurate, isocetyl stearate, isopropyl isostearate, isopropyl myristate, isodecyl oleate, glyceryl tri(2-ethylhexanoate), pentaerythrithyl tetra(2-ethylhexanoate), 2-ethylhexyl succinate, diethyl sebacate, and mixtures thereof.

As examples of artificial triglycerides, mention may be made of, for example, capryl caprylyl glycerides, glyceryl trimyristate, glyceryl tripalmitate, glyceryl trilinolenate, glyceryl trilaurate, glyceryl tricaprate, glyceryl tricaprylate, glyceryl tri(caprate/caprylate) and glyceryl tri(caprate/caprylate/linolenate).

As examples of silicone oils, mention may be made of, for example, linear organopolysiloxanes such as dimethylpolysiloxane, methylphenylpolysiloxane, methylhydrogenpolysiloxane, and the like; cyclic organopolysiloxanes such as cyclohexasiloxane, octamethylcyclotetrasiloxane, decamethylcyclopentasiloxane, dodecamethylcyclohexasiloxane, and the like; and mixtures thereof.

Preferably, silicone oil is chosen from liquid polydialkylsiloxanes, especially liquid polydimethylsiloxanes (PDMS) and liquid polyorganosiloxanes comprising at least one aryl group.

These silicone oils may also be organomodified. The organomodified silicones that can be used in accordance with the present invention are silicone oils as defined above and comprise in their structure one or more organofunctional groups attached via a hydrocarbon-based group.

Organopolysiloxanes are defined in greater detail in Walter Noll's Chemistry and Technology of Silicones (1968), Academic Press. They may be volatile or non-volatile.

When they are volatile, the silicones are more particularly chosen from those having a boiling point of between 60° C. and 260° C., and even more particularly from:
(i) cyclic polydialkylsiloxanes comprising from 3 to 7 and preferably 4 to 5 silicon atoms. These are, for example, octamethylcyclotetrasiloxane sold in particular under the name Volatile Silicone® 7207 by Union Carbide or Silbione® 70045 V2 by Rhodia, decamethylcyclopentasiloxane sold under the name Volatile Silicone® 7158 by Union Carbide, Silbione® 70045 V5 by Rhodia, and dodecamethylcyclopentasiloxane sold under the name Silsoft 1217 by Momentive Performance Materials, and mixtures thereof. Mention may also be made of cyclocopolymers of the type such as dimethylsiloxane/methylalkylsiloxane, such as Silicone Volatile® FZ 3109 sold by the company Union Carbide, of formula:

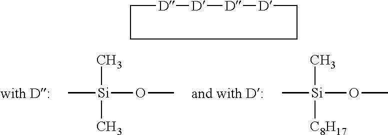

Mention may also be made of mixtures of cyclic polydialkylsiloxanes with organosilicon compounds, such as the mixture of octamethylcyclotetrasiloxane and tetratrimethylsilylpentaetythritol (50/50) and the mixture of octamethylcyclotetrasiloxane and oxy-1,1′-bis(2,2,2′,2′,3,3′-hexatrimethylsilyloxy)neopentane;
(ii) linear volatile polydialkylsiloxanes containing 2 to 9 silicon atoms and having a viscosity of less than or equal to 5×10⁻⁶ m²/s at 25° C. An example is decamethyltetrasiloxane sold in particular under the name SH 200 by the company Toray Silicone. Silicones belonging to this category are also described in the article published in Cosmetics and Toiletries, Vol. 91, Jan. 76, pp. 27-32, Todd & Byers, Volatile Silicone Fluids for Cosmetics. The viscosity of the silicones is measured at 25° C. according to ASTM standard 445 Appendix C.

Non-volatile polydialkylsiloxanes may also be used. These non-volatile silicones are more particularly chosen from polydialkylsiloxanes, among which mention may be made mainly of polydimethylsiloxanes containing trimethylsilyl end groups.

Among these polydialkylsiloxanes, mention may be made, in a non-limiting manner, of the following commercial products:
the Silbione® oils of the 47 and 70 047 series or the Mirasil® oils sold by Rhodia, for instance the oil 70 047 V 500 000;
the oils of the Mirasil® series sold by the company Rhodia;
the oils of the 200 series from the company Dow Corning, such as DC200 with a viscosity of 60 000 mm²/s;
the Viscasil® oils from General Electric and certain oils of the SF series (SF 96, SF 18) from General Electric.

Mention may also be made of polydimethylsiloxanes containing dimethylsilanol end groups known under the name dimethiconol (CTFA), such as the oils of the 48 series from the company Rhodia.

Among the silicones containing aryl groups are polydiarylsiloxanes, especially polydiphenylsiloxanes and polyalkylarylsiloxanes. Examples that may be mentioned include the products sold under the following names:
the Silbione® oils of the 70 641 series from Rhodia;
the oils of the Rhodorsil® 70 633 and 763 series from Rhodia;
the oil Dow Corning 556 Cosmetic Grade Fluid from Dow Corning;
the silicones of the PK series from Bayer, such as the product PK20;
certain oils of the SF series from General Electric, such as SF 1023, SF 1154, SF 1250 and SF 1265.

The organomodified liquid silicones may especially contain polyethyleneoxy and/or polypropyleneoxy groups. Mention may thus be made of the silicone KF-6017 proposed by Shin-Etsu, and the oils Silwet® L722 and L77 from the company Union Carbide.

Hydrocarbon oils may be chosen from:
linear or branched, optionally cyclic, C₆-C₁₆ lower alkanes. Examples that may be mentioned include hexane, undecane, dodecane, tridecane, and isoparaffins, for instance isohexadecane, isododecane and isodecane; and
linear or branched hydrocarbons containing more than 16 carbon atoms, such as liquid paraffins, liquid petroleum jelly, polydecenes and hydrogenated polyisobutenes such as Parleam®, and squalane.

As preferable examples of hydrocarbon oils, mention may be made of, for example, linear or branched hydrocarbons such as isohexadecane, isododecane, squalane, mineral oil(e.g., liquid paraffin), paraffin, vaseline or petrolatum, naphthalenes, and the like; hydrogenated polyisobutene, isoeicosan, and decene/butene copolymer; and mixtures thereof.

The term “fatty” in the fatty alcohol means the inclusion of a relatively large number of carbon atoms. Thus, alcohols which have 4 or more, preferably 6 or more, and more preferably 12 or more carbon atoms are encompassed within the scope of fatty alcohols. The fatty alcohol may be saturated or unsaturated. The fatty alcohol may be linear or branched.

The fatty alcohol may have the structure R—OH wherein R is chosen from saturated and unsaturated, linear and branched radicals containing from 4 to 40 carbon atoms, preferably from 6 to 30 carbon atoms, and more preferably from 12 to 20 carbon atoms. In at least one embodiment, R may be chosen from C₁₂-C₂₀ alkyl and C₁₂-C₂₀ alkenyl groups. R may be or may not be substituted with at least one hydroxyl group.

As examples of the fatty alcohol, mention may be made of lauryl alcohol, cetyl alcohol, stearyl alcohol, isostearyl alcohol, behenyl alcohol, undecylenyl alcohol, myristyl alcohol, octyldodecanol, hexyldecanol, oleyl alcohol, linoleyl alcohol, palmitoleyl alcohol, arachidonyl alcohol, erucyl alcohol, and mixtures thereof.

It is preferable that fatty alcohol be a saturated fatty alcohol.

Thus, the fatty alcohol may be selected from straight or branched, saturated or unsaturated C₆-C₃₀ alcohols, preferably straight or branched, saturated C₆-C₃₀ alcohols, and more preferably straight or branched, saturated C₁₂-C₂₀ alcohols.

The term “saturated fatty alcohol” here means an alcohol having a long aliphatic saturated carbon chain. It is preferable that the saturated fatty alcohol be selected from any linear or branched, saturated C₆-C₃₀ fatty alcohols. Among the linear or branched, saturated C₆-C₃₀ fatty alcohols, linear or branched, saturated C₁₂-C₂₀ fatty alcohols may preferably be used. Any linear or branched, saturated C₁₆-C₂₀ fatty alcohols may be more preferably used. Branched C₁₆-C₂₀ fatty alcohols may be even more preferably used.

As examples of saturated fatty alcohols, mention may be made of lauryl alcohol, cetyl alcohol, stearyl alcohol, isostearyl alcohol, behenyl alcohol, undecylenyl alcohol, myristyl alcohol, octyldodecanol, hexyldecanol, and mixtures thereof. In one embodiment, cetyl alcohol, stearyl alcohol, octyldodecanol, hexyldecanol, or a mixture thereof (e.g., cetearyl alcohol) as well as behenyl alcohol, can be used as a saturated fatty alcohol.

According to at least one embodiment, the fatty alcohol used in the composition according to the present invention is preferably chosen from octyldodecanol, hexyldecanol and mixtures thereof.

It is preferable that the (a) oil be chosen from ester oils and fatty alcohols.

It may be preferable that the (a) oil be chosen from oils with molecular weight below 600 g/mol.

The amount in the composition according to the present invention of the (a) oil is not limited, and may range from 0.1 to 35% by weight, preferably from 0.5 to 30% by weight, and more preferably from 1 to 25% by weight, relative to the total weight of the composition.

### [Polyglyceryl Fatty Acid Ester]

The composition according to the present invention comprises at least one polyglyceryl fatty acid ester. A single type of polyglyceryl fatty acid ester may be used, but two or more different types of polyglyceryl fatty acid ester may be used in combination.

It is preferable that the (b) polyglyceryl fatty acid ester have a polyglycerol moiety derived from 2 to 10 glycerols, more preferably from 2 to 8 glycerols, and further more preferably 2 to 6 glycerols.

The (b) polyglyceryl fatty acid ester may have an HLB (Hydrophilic Lipophilic Balance) value of from 7.0 to 14.0, preferably from 8.0 to 13.5, and more preferably from 10.0 to 13.0. If two or more polyglyceryl fatty acid esters are used, the HLB value is determined by the weight average of the HLB values of all the polyglyceryl fatty acid esters.

The (b) polyglyceryl fatty acid ester may be chosen from the mono, di and tri esters of saturated or unsaturated acid, preferably saturated acid, including 2 to 30 carbon atoms, preferably 6 to 30 carbon atoms, and more preferably 8 to 30 carbon atoms, such as lauric acid, oleic acid, stearic acid, isostearic acid, capric acid, caprylic acid, and myristic acid.

The (b) polyglyceryl fatty acid ester may be selected from the group consisting of PG2 caprylate, PG2 sesquicaprylate, PG2 dicaprylate, PG2 tricaprylate, PG2 caprate, PG2 sesquicaprate, PG2 dicaprate, PG2 tricaprate, PG2 laurate, PG2 sesquilaurate, PG2 dilaurate, PG2 trilaurate, PG2 myristate, PG2 sesquimyristate, PG2 dimyristate, PG2 trimyristate, PG2 stearate, PG2 sesquistearate, PG2 distearate, PG2 tristearate, PG2 isostearate, PG2 sesquiisostearate, PG2 diisostearate, PG2 triisostearate, PG2 oleate, PG2 sesquioleate, PG2 dioleate, PG2 trioleate, PG3 caprylate, PG3 sesquicaprylate, PG3 dicaprylate, PG3 tricaprylate, PG3 caprate, PG3 sesquicaprate, PG3 dicaprate, PG3 tricaprate, PG3 laurate, PG3 sesquilaurate, PG3 dilaurate, PG3 trilaurate, PG3 myristate, PG3 sesquimyristate, PG3 dimyristate, PG3 trimyristate, PG3 stearate, PG3 sesquistearate, PG3 distearate, PG3 tristearate, PG3 isostearate, PG3 sesquiisostearate, PG3 diisostearate, PG3 triisostearate, PG3 oleate, PG3 sesquioleate, PG3 dioleate, PG3 trioleate, PG4 caprylate, PG4 sesquicaprylate, PG4 dicaprylate, PG4 tricaprylate, PG4 caprate, PG4 sesquicaprate, PG4 dicaprate, PG4 tricaprate, PG4 laurate, PG4 sesquilaurate, PG4 dilaurate, PG4 trilaurate, PG4 myristate, PG4 sesquimyristate, PG4 dimyristate, PG4 trimyristate, PG4 stearate, PG4 sesquistearate, PG4 distearate, PG4 tristearate, PG4 isostearate, PG4 sesquiisostearate, PG4 diisostearate, PG4 triisostearate, PG4 oleate, PG4 sesquioleate, PG4 dioleate, PG4 trioleate, PG5 caprylate, PG5 sesquicaprylate, PG5 dicaprylate, PG5 tricaprylate, PG5 tetracaprylate, PG5 caprate, PG5 sesquicaprate, PG5 dicaprate, PG5 tricaprate, PG5 tetracaprate, PG5 laurate, PG5 sesquilaurate, PG5 dilaurate, PG5 trilaurate, PG5 tetralaurate, PG5 myristate, PG5 sesquimyristate, PG5 dimyristate, PG5 trimyristate, PG5 tetramyristate, PG5 stearate, PG5 sesquistearate, PG5 distearate, PG5 tristearate, PG5 tetrastearate, PG5 isostearate, PG5 sesquiisostearate, PG5 diisostearate, PG5 triisostearate, PG5 tetraisostearate, PG5 oleate, PG5 sesquioleate, PG5 dioleate, PG5 trioleate, PG5 tetraoleate, PG6 caprylate, PG6 sesquicaprylate, PG6 dicaprylate, PG6 tricaprylate, PG6 tetracaprylate, PG6 pentacaprylate, PG6 caprate, PG6 sesquicaprate, PG6 dicaprate, PG6 tricaprate, PG6 tetracaprate, PG6 pentacaprate, PG6 laurate, PG6 sesquilaurate, PG6 dilaurate, PG6 trilaurate, PG6 tetralaurate, PG6 pentalaurate, PG6 myristate, PG6 sesquimyristate, PG6 dimyristate, PG6 trimyristate, PG6 tetramyristate, PG6 pentamyristate, PG6 stearate, PG6 sesquistearate, PG6 distearate, PG6 tristearate, PG6 tetrastearate, PG6 pentastearate, PG6 isostearate, PG6 sesquiisostearate, PG6 diisostearate, PG6 triisostearate, PG6 tetraisostearate, PG6 pentaisostearate, PG6 oleate, PG6 sesquioleate, PG6 dioleate, PG6 trioleate, PG6 tetraoleate, PG6 pentaoleate, PG10 caprylate, PG10 sesquicaprylate, PG10 dicaprylate, PG10 tricaprylate, PG10 tetracaprylate, PG10 pentacaprylate, PG10 hexacaprylate, PG10 caprate, PG10 sesquicaprate, PG10 dicaprate, PG10 tricaprate, PG10 tetracaprate, PG10 pentacaprate, PG10 hexacaprate, PG10 laurate, PG10 sesquilaurate, PG10 dilaurate, PG10 trilaurate, PG10 tetralaurate, PG10 pentalaurate, PG10 hexalaurate, PG10 myristate, PG10 sesquimyristate, PG10 dimyristate, PG10 trimyristate, PG10 tetramyristate, PG10 pentamyristate, PG10 hexamyristate, PG10 stearate, PG10 sesquistearate, PG10 distearate, PG10 tristearate, PG10 tetrastearate, PG10 pentastearate, PG10 hexastearate, PG10 isostearate, PG10 sesquiisostearate, PG10 diisostearate, PG10 triisostearate, PG10 tetraisostearate, PG10 pentaisostearate, PG10 hexaisostearate, PG10 oleate, PG10 sesquioleate, PG10 dioleate, PG10 trioleate, PG10 tetraoleate, PG10 pentaoleate, and PG10 hexaoleate.

It is preferable that the (b) polyglyceryl fatty acid ester be chosen from:
polyglyceryl monolaurate comprising 2 to 6 glycerol units,
polyglyceryl mono(iso)stearate comprising 2 to 6 glycerol units,
polyglyceryl monooleate comprising 2 to 6 glycerol units, and
polyglyceryl dioleate comprising 2 to 6 glycerol units.

In one embodiment, the (b) polyglyceryl fatty acid ester raw material may be chosen from a mixture of polyglyceryl fatty acid esters, preferably with a polyglyceryl moiety derived from 2 to 10 glycerins, more preferably 2 to 6 glycerins, wherein the mixture preferably comprises 30% by weight or more of a polyglyceryl fatty acid ester with a polyglyceryl moiety consisting of 2 to 6 glycerins.

It may be preferable than the (b) polyglyceryl fatty acid ester raw material comprise esters of a fatty acid and polyglycerine containing 70% or more of polyglycerine whose polymerization degree is 2 or more, preferably esters of a fatty acid and polyglycerine containing equal to or more than 60% of polyglycerine whose polymerization degree is between 2 and 6, and more preferably esters of a fatty acid and polyglycerine containing equal to or more than 30% of polyglycerine whose polymerization degree is 2 to 6.

The amount in the composition according to the present invention of the (b) polyglyceryl fatty acid ester is not limited, and may range from 0.1 to 20% by weight, preferably from 0.5 to 15% by weight, and more preferably from 1 to 10% by weight, relative to the total weight of the composition.

### [Silicone Elastomer]

The composition according to the present invention comprises at least one silicone elastomer. A single type of silicone elastomer may be used, but two or more different types of silicone elastomer may be used in combination.

In a preferred embodiment, the (c) silicone elastomer is a non-emulsifying silicon elastomer.

The (c) silicone elastomer, preferably non-emulsifying silicon elastomer, may be in the form of a powder. The (c) silicone elastomer may be added to the composition in the form of either a powder or a gel in which the elastomer is dispersed in an oil such as a silicone oil and a hydrocarbon oil wherein the elastomer concentration may be from 5 to 60% by weight, preferably from 10 to 30% by weight, relative to the total weight of the gel.

The “silicone elastomer” or “organopolysiloxane elastomer” makes it possible to remove or reduce the stickiness caused by the (b) polyglyceryl fatty acid ester. It also provides a very soft and mattifying feel after application, which is especially advantageous for application to the skin.

The expression “silicone elastomer” or “organopolysiloxane elastomer” means a flexible, deformable organopolysiloxane having viscoelastic properties and especially the consistency of a sponge or a flexible sphere. Its modulus of elasticity is such that this material withstands deformation and has limited stretchability and contractibility. This material is capable of regaining its original shape after stretching.

It is more particularly a crosslinked organopolysiloxane elastomer.

Thus, the organopolysiloxane elastomer may be obtained by a crosslinking addition reaction of diorganopolysiloxane containing at least one hydrogen bonded to silicon and of diorganopolysiloxane containing ethylenically unsaturated groups bonded to silicon, especially in the presence of a platinum catalyst; or by a dehydrogenation crosslinking condensation reaction between a diorganopolysiloxane containing hydroxyl end groups and a diorganopolysiloxane containing at least one hydrogen bonded to silicon, especially in the presence of an organotin; or by a crosslinking condensation reaction of a diorganopolysiloxane containing hydroxyl end groups and of a hydrolysable organopolysilane; or by thermal crosslinking of organopolysiloxane, especially in the presence of an organoperoxide catalyst; or by crosslinking of organopolysiloxane via high-energy radiation such as gamma rays, ultraviolet rays or an electron beam.

Preferably, the organopolysiloxane elastomer is obtained by a crosslinking addition reaction of (A) diorganopolysiloxane containing at least two hydrogens each bonded to silicon, and of (B) diorganopolysiloxane containing at least two ethylenically unsaturated groups bonded to silicon, especially in the presence of (C) a platinum catalyst, as described, for instance, in patent application EP-A-295 886.

In particular, the organopolysiloxane elastomer may be obtained by reaction of a dimethylpolysiloxane containing dimethylvinylsiloxy end groups and of methylhydrogenpolysiloxane containing trimethylsiloxy end groups, in the presence of a platinum catalyst.

Compound (A) is the base reagent for the formation of organopolysiloxane elastomer, and the crosslinking is performed by an addition reaction of compound (A) with compound (B) in the presence of catalyst (C).

Compound (A) is in particular an organopolysiloxane containing at least two hydrogen atoms bonded to different silicon atoms in each molecule.

Compound (A) may have any molecular structure, especially a linear-chain or branched-chain structure or a cyclic structure.

Compound (A) may have a viscosity at 25° C. ranging from 1 to 50 000 centistokes, especially so as to be miscible with compound (B).

The organic groups bonded to the silicon atoms of compound (A) may be alkyl groups such as methyl, ethyl, propyl, butyl, octyl; substituted alkyl groups such as 2-phenylethyl, 2-phenylpropyl or 3,3,3-trifluoropropyl; aryl groups such as phenyl, tolyl, xylyl; substituted aryl groups such as phenylethyl; and substituted monovalent hydrocarbon-based groups such as an epoxy group, a carboxylate ester group or a mercapto group.

Compound (A) may thus be chosen from methylhydrogenpolysiloxanes containing trimethylsiloxy end groups, dimethylsiloxane-methylhydrosiloxane copolymers containing trimethylsiloxy end groups, and dimethylsiloxane-methylhydrosiloxane cyclic copolymers.

Compound (B) is advantageously a diorganopolysiloxane containing at least two lower alkenyl groups (for example C₂-C₄); the lower alkenyl group may be chosen from vinyl, ally! and propenyl groups. These lower alkenyl groups may be located in any position of the organopolysiloxane molecule, but are preferably located at the ends of the organopolysiloxane molecule. The organopolysiloxane (B) may have a branched-chain, linear-chain, cyclic or network structure, but the linear-chain structure is preferred. Compound (B) may have a viscosity ranging from the liquid state to the gum state. Preferably, compound (B) has a viscosity of at least 100 centistokes at 25° C.

Besides the above-mentioned alkenyl groups, the other organic groups bonded to the silicon atoms in compound (B) may be alkyl groups such as methyl, ethyl, propyl, butyl or octyl; substituted alkyl groups such as 2-phenylethyl, 2-phenylpropyl or 3,3,3-trifluoropropyl; aryl groups such as phenyl, tolyl or xylyl; substituted aryl groups such as phenylethyl; and substituted monovalent hydrocarbon-based groups such as an epoxy group, a carboxylate ester group or a mercapto group.

The organopolysiloxanes (B) may be chosen from methylvinylpolysiloxanes, methylvinylsiloxane-dimethylsiloxane copolymers, dimethylpolysiloxanes containing dimethylvinylsiloxy end groups, dimethylsiloxane-methylphenylsiloxane copolymers containing dimethylvinylsiloxy end groups, dimethylsiloxane-diphenylsiloxane-methylvinylsiloxane copolymers containing dimethylvinylsiloxy end groups, dimethylsiloxane-methylvinylsiloxane copolymers containing trimethylsiloxy end groups, dimethylsiloxane-methylphenylsiloxane-methylvinylsiloxane copolymers containing trimethylsiloxy end groups, methyl(3,3,3-trifluoropropyl)polysiloxanes containing dimethylvinylsiloxy end groups, and dimethylsiloxane-methyl(3,3,3-trifluoropropyl)siloxane copolymers containing dimethylvinylsiloxy end groups.

In particular, the organopolysiloxane elastomer may be obtained by reaction of dimethylpolysiloxane containing dimethylvinylsiloxy end groups and of methylhydrogenpolysiloxane containing trimethylsiloxy end groups, in the presence of a platinum catalyst.

Advantageously, the sum of the number of ethylenic groups per molecule in compound (B) and of the number of hydrogen atoms bonded to silicon atoms per molecule in compound (A) is at least 5.

It is advantageous for compound (A) to be added in an amount such that the molecular ratio between the total amount of hydrogen atoms bonded to silicon atoms in compound (A) and the total amount of all the ethylenically unsaturated groups in compound (B) is within the range from 1.5/1 to 20/1.

Compound (C) is the catalyst for the crosslinking reaction, and is especially chloroplatinic acid, chloroplatinic acid-olefin complexes, chloroplatinic acid-alkenylsiloxane complexes, chloroplatinic acid-diketone complexes, platinum black and platinum on a support.

The catalyst (C) is preferably added in an amount of from 0.1 to 1000 parts by weight and better still from 1 to 100 parts by weight, as clean platinum metal, per 1000 parts by weight of the total amount of compounds (A) and (B).

The elastomer is advantageously a non-emulsifying elastomer.

The term “non-emulsifying” defines organopolysiloxane elastomers not containing any hydrophilic chains, and in particular not containing any polyoxyalkylene units (especially polyoxyethylene or polyoxypropylene) or any polyglyceryl units. Thus, according to one particular embodiment of the present invention, the composition of the present invention comprises an organopolysiloxane elastomer that is free of polyoxyalkylene units and polyglyceryl units.

Non-emulsifying elastomers are especially described in patents EP 242 219, EP 285 886 and EP 765 656 and in patent application JP-A-61-194 009.

Non-emulsifying elastomers that may be used more particularly include those sold under the names KSG-6, KSG-15, KSG-16, KSG-18, KSG-19, KSG-41, KSG-42, KSG-43 and KSG-44 by the company Shin-Etsu, DC 9040 and DC 9041 by the company Dow Corning, and SFE 839 by the company General Electric.

Spherical non-emulsifying elastomers that may be used include those sold under the names DC 9040, DC 9041, DC 9240, DC 9509, DC 9505 and DC 9506 by the company Dow Corning.

In an embodiment, the organopolysiloxane elastomer particles are conveyed in the form of a gel formed from an elastomeric organopolysiloxane included in at least one hydrocarbon-based oil and/or one silicone oil. In these gels, the organopolysiloxane particles are often non-spherical particles.

As preferred non-emulsifying silicone elastomers in gel form, we may cite the INCI Name products Dimethicone crosspolymers, for example Dimethicone crosslinked with C₃-C₂₀ alkyl group, such as DC9041, DC9240, and DC9045 from Dow Corning, Polysilicone-11 and Cyclohexasiloxane (Polysilicone-11 swelled with Cyclohexasiloxane) such as Gransil RPS-D6 from Grant Industries, Polysilicone-11 and isododecane (Polysilicone-11 swelled with Cyclohexasiloxane) such as Gransil PC-12 from Grant Industries (weight ratio of polysilicone-11:isododecane is 13:87), and Dimethicone/Vinyldimethicone crosspolymer andDimethicone (Dimethicone/Vinyldimethicone crosspolymer swelled with Dimethicone) such as KSG16 from Shin Etsu (weight ratio of dimethicone/vinyldimethicone crosspolymer: dimethicone is 24:76).

In another embodiment, the organopolysiloxane elastomer particles are conveyed in the form of a powder.

As preferred non-emulsifying silicone elastomers in powder form, we may cite the INCI Name products Dimethicone/Vinyldimethicone crosspolymer such as DC9506 and DC9701 from Dow Corning and KSG6 from Shin Etsu.

In another embodiment, the composition of the present invention comprises at least one silicone elastomer powder coated with a silicone resin. The silicone elastomer powder is spherical and may be obtained especially via the processes for synthesizing non-emulsifying elastomers described above. The silicone elastomer powder is coated with silicone resin.

According to one preferred embodiment, the silicone resin may be a silsesquioxane resin, as described, for example, in patent U.S. Pat. No. 5,538,793, the content of which is incorporated herein by way of reference. Such elastomer powders coated with silicone resin are especially sold under the names KSP-100, KSP-101, KSP-102, KSP-103, KSP-104 and KSP-105 by the company Shin-Etsu. Such powders correspond to the INCI name dimethicone silsesquioxane crosspolymer, and in particular vinyl dimethicone/methicone silsesquioxane crosspolymer. As a preferred elastomer powder coated with silicone resin, we may use KSP100.

The silicone elastomer particles may have a JIS-A hardness of less than or equal to 80 (especially ranging from 5 to 80) and preferably less than or equal to 65 (especially ranging from 5 to 65). The JIS-A hardness is measured according to the method JIS K 6301 (1995) established by the Japanese Industrial Standards Committee.

In particular, the silicone elastomer particles may have a mean size ranging from 0.1 to 500 μm, preferably from 3 to 200 μm and better still from 10 to 20 μm. These particles may be of spherical, flat or amorphous shape, and preferably of spherical shape.

The amount of the (c) silicone elastomer is not limited, and may range from 0.05 to 15% by weight, preferably from 0.1 to 10% by weight, more preferably from 0.2 to 5% by weight, and even more preferably 0.3 to 4% by weight, relative to the total weight of the composition.

### [Polysaccharide]

The composition according to the present invention comprises at least one polysaccharide. A single type of polysaccharide may be used, but two or more different types of polysaccharide may be used in combination.

The (d) polysaccharide can provide the composition according to the present invention with stability, in particular stability over time and/or under elevated temperature.

The polysaccharides are, for example, chosen from glucans, modified and unmodified starches (such as those derived, for example, from cereals, for instance wheat, corn, or rice, from vegetables, for instance yellow peas, and tubers, for instance potatoes or cassava), amylose, amylopectin, glycogen, dextrans, celluloses, and derivatives thereof (e.g., methylcelluloses, hydroxyalkylcelluloses, ethyl hydroxyethylcelluloses, and carboxymethylcelluloses), mannans, xylans, lignins, arabans, galactans, galacturonans, chitin, chitosans, glucuronoxylans, arabinoxylans, xyloglucans, glucomannans, pectic acids, and pectins, alginic acid and alginates, arabinogalactans, carrageenans, agars, glycosaminoglucans, gum arabics, gum tragacanths, ghatti gums, karaya gums, carob gums, galactomannans, such as guar gums, and nonionic derivatives thereof (e.g., hydroxypropyl guar), sclerotium gums, xanthan gums, and mixtures thereof.

For example, the polysaccharides that may be used are chosen from those described, for example, in “Encyclopedia of Chemical Technology”, Kirk-Othmer, Third Edition, 1982, volume 3, pp. 896-900, and volume 15, pp. 439-458, in “Polymers in Nature” by E. A. MacGregor and C. T. Greenwood, published by John Wiley & Sons, Chapter 6, pp. 240-328, 1980, and in “Industrial Gums-Polysaccharides and their Derivatives”, edited by Roy L. Whistler, Second Edition, published by Academic Press Inc., the content of these three publications being entirely incorporated by reference.

For example, starches, guar gums, celluloses, and derivatives thereof can be used.

Among the starches that may be used, mention may be made, for example, of macromolecules in the form of polymers comprising base units which are anhydroglucose units. The number of these units and their assembly make it possible to distinguish between amylose (linear polymer) and amylopectin (branched polymer). The relative proportions of amylose and amylopectin, as well as their degree of polymerization, can vary according to the botanical origin of the starches.

The molecules of the starches used may have cereals or tubers as their botanical origin. Thus, the starches can be, for example, chosen from maize, rice, cassava, tapioca, barley, potato, wheat, sorghum, and pea starches.

Starches generally exist in the form of a white powder, insoluble in cold water, whose elementary particle size ranges from 3 to 100 microns.

The starches may be optionally C₁-C₆ hydroxyalkylated or C₁-C₆ acylated (such as acetylated). The starches may have also undergone heat treatments.

Distarch phosphates or compounds rich in distarch phosphate, such as the product provided under the references PREJEL VA-70-T AGGL (gelatinized hydroxypropylated cassava distarch phosphate) or PREJEL TK1 (gelatinized cassava distarch phosphate) or PREJEL 200 (gelatinized acetylated cassava distarch phosphate) by the company AVEBE, may also be used.

The guar gums can be modified or unmodified.

The unmodified guar gums are, for example, the products sold under the name Vidogum GH 175 by the company Unipectine and under the names Meypro-Guar 50 and Jaguar C by the company Meyhall.

The modified nonionic guar gums are, for example, modified with C₁-C₆ hydroxyalkyl groups.

Among the hydroxyalkyl groups that may be mentioned, for example, are hydroxymethyl, hydroxyethyl, hydroxypropyl, and hydroxybutyl groups.

These guar gums are well known in the prior art and can be prepared, for example, by reacting the corresponding alkene oxides such as propylene oxides, with the guar gum so as to obtain a guar gum modified with hydroxypropyl groups.

The degree of hydroxyalkylation, which corresponds to the number of alkylene oxide molecules consumed by the number of free hydroxyl functions present on the guar gum, may, for example, range from 0.4 to 1.2.

Such nonionic guar gums optionally modified with hydroxyalkyl groups are sold, for example, under the trade names Jaguar HP8, Jaguar HP60, Jaguar HP120, Jaguar DC 293, and Jaguar HP 105 by the company Rhodia Chimie (Meyhall) or under the name Galactasol 4H_{4FD2} by the company Aqualon.

Among the celluloses and cellulose derivatives that are used are, for example, hydroxypropylmethylcellulose, hydroxyethylcellulose, and hydroxypropylcelluloses, as well as hydrophobicized hydroxypropylmethylcellulose. Mention may be made of the products sold under the names Klucel E F, Klucel H, Klucel L H F, Klucel M F, and Klucel G by the company Aqualon.

It is preferable that the (d) polysaccharide be derived from microorganisms.

As examples of the polysaccharides derived from microorganisms, mention may be made of cardollan, xanthan gum, Jellan gum, dextran, pullulan, sclerotium gum, and mixtures thereof. It may be preferable to use sclerotium gum and/or xanthan gum.

The amount of the (d) polysaccharide is not limited, and may range from 0.01 to 10% by weight, preferably from 0.05 to 5% by weight, more preferably from 0.1 to 1% by weight, and even more preferably 0.2 to 0.8% by weight, relative to the total weight of the composition.

### [Water]

The composition according to the present invention may comprise (e) water.

The amount of (e) water is not limited, and may be from 40 to 95% by weight, preferably from 45 to 90% by weight, and more preferably 50 to 85% by weight, relative to the total weight of the composition.

### [Additional Surfactant]

The composition according to the present invention may further comprise at least one additional surfactant different from the above (b) polyglyceryl fatty acid ester. A single type of additional surfactant may be used, but two or more different types of additional surfactant may be used in combination. The (f) additional surfactant may preferably be selected from cationic surfactants, anionic surfactants, and amphoteric surfactants, and more preferably from anionic surfactants.

### (f1) Anionic Surfactants

According to the present invention, the type of anionic surfactant is not limited. It is preferable that the anionic surfactant be selected from the group consisting of (C₆-C₃₀) alkyl sulfates, (C₆-C₃₀) alkyl ether sulfates, (C₆-C₃₀) alkylamido ether sulfates, alkylaryl polyether sulfates, monoglyceride sulfates; (C₆-C₃₀) alkylsulfonates, (C₆-C₃₀) alkylamide sulfonates, (C₆-C₃₀) alkylaryl sulfonates, α-olefin sulfonates, paraffin sulfonates; (C₆-C₃₀) alkyl phosphates; (C₆-C₃₀) alkyl sulfosuccinates, (C₆-C₃₀) alkyl ether sulfosuccinates, (C₆-C₃₀) alkylamide sulfosuccinates; (C₆-C₃₀) alkyl sulfoacetates; (C₆-C₂₄) acyl sarcosinates; (C₆-C₂₄) acyl glutamates; (C₆-C₃₀) alkylpolyglycoside carboxylic ethers; (C₆-C₃₀) alkylpolyglycoside sulfosuccinates; (C₆-C₃₀) alkyl sulfosuccinamates; (C₆-C₂₄) acyl isethionates; N—(C₆-C₂₄) acyl taurates; C₆-C₃₀ fatty acid salts; coconut oil acid salts or hydrogenated coconut oil acid salts; (C₈-C₂₀) acyl lactylates; (C₆-C₃₀) alkyl-D-galactoside uronic acid salts; polyoxyalkylenated (C₆-C₃₀) alkyl ether carboxylic acid salts; polyoxyalkylenated (C₆-C₃₀) alkylaryl ether carboxylic acid salts; polyoxyalkylenated (C₆-C₃₀) alkylamido ether carboxylic acid salts; and polyoxyalkylenated (C₆-C₃₀) alkyl ether phosphates.

It is more preferable that the anionic surfactant be selected from salts of C₆-C₃₀ acyl glutamates such as sodium stearoyl glutamate, (C₆-C₂₄) acyl taurates such as sodium methyl stearoyl taurate, and mixtures thereof.

In at least one embodiment, the anionic surfactants are in the form of salts such as salts of alkali metals, for instance sodium; salts of alkaline-earth metals, for instance magnesium; ammonium salts; amine salts; and amino alcohol salts. Depending on the conditions, they may also be in acid form.

It should be noted that the alkyl or acyl radicals of these various compounds can contain from 12 to 20 carbon atoms. Moreover, for instance, the aryl radical can be chosen from a phenyl or benzyl group.

Furthermore, the polyoxyalkylenated anionic surfactants can, for example, comprise from 2 to 50 alkylene oxide, for instance ethylene oxide, groups.

In accordance with at least one embodiment of the present disclosure, the anionic surfactant can be chosen from stearic acid, dicetyl phosphate and ceteth-10 phosphate.

### (f2) Amphoteric Surfactants

According to the present invention, the type of amphoteric surfactant is not limited. The amphoteric or zwitterionic surfactants can be, for example (non-limiting list), amine derivatives such as aliphatic secondary or tertiary amines, and optionally quaternized amine derivatives, in which the aliphatic radical is a linear or branched chain comprising 8 to 22 carbon atoms and containing at least one water-solubilizing anionic group (for example, carboxylate, sulfonate, sulfate, phosphate or phosphonate).

The amphoteric surfactant may preferably be selected from the group consisting of betaines and amidoaminecarboxylated derivatives.

The betaine-type amphoteric surfactant is preferably selected from the group consisting of alkylbetaines, alkylamidoalkylbetaines, sulfobetaines, phosphobetaines, and alkylamidoalkylsulfobetaines, in particular, (C₈-C₂₄) alkylbetaines, (C₈-C₂₄) alkylamido (C₁-C₈) alkylbetaines, sulfobetaines, and (C₈-C₂₄) alkylamido (C₁-C₈) alkylsulfobetaines. In one embodiment, the amphoteric surfactants of betaine type are chosen from (C₈-C₂₄) alkylbetaines, (C₈-C₂₄) alkylamido(C₁-C₈) alkylsulfobetaines, sulfobetaines, and phosphobetaines.

Non-limiting examples that may be mentioned include the compounds classified in the CTFA dictionary, 9th edition, 2002, under the names cocobetaine, laurylbetaine, cetylbetaine, coco/oleamidopropylbetaine, cocamidopropylbetaine, palmitamidopropylbetaine, stearamidopropylbetaine, cocamidoethylbetaine, cocamidopropylhydroxysultaine, oleamidopropylhydroxysultaine, cocohydroxysultaine, laurylhydroxysultaine, and cocosultaine, alone or as mixtures.

The betaine-type amphoteric surfactant is preferably an alkylbetaine and an alkylamidoalkylbetaine, in particular cocobetaine and cocamidopropylbetaine.

Among the amidoaminecarboxylated derivatives, mention may be made of the products sold under the name Miranol, as described in U.S. Pat. Nos. 2,528,378 and 2,781,354 and classified in the CTFA dictionary, 3rd edition, 1982 (the disclosures of which are incorporated herein by reference), under the names Amphocarboxyglycinates and Amphocarboxypropionates, with the respective structures:

R₁—CONHCH₂CH₂—N⁺(R₂)(R₃)(CH₂COO⁻)

in which:
R₁ denotes an alkyl radical of an acid R₁—COOH present in hydrolysed coconut oil, a heptyl, nonyl or undecyl radical,
R₂ denotes a beta-hydroxyethyl group, and
R₃ denotes a carboxymethyl group; and

R₁′—CONHCH₂CH₂—N(B)(C)

in which:
B represents —CH₂CH₂OX′,
C represents —(CH₂)_{z}—Y′, with z=1 or 2,
X′ denotes a —CH₂CH₂—COOH group, —CH₂—COOZ′, —CH₂CH₂—COOH, —CH₂CH₂—COOZ′ or a hydrogen atom,
Y′ denotes —COOH, —COOZ′, —CH₂—CHOH—SO₃Z′ or a —CH₂—CHOH—SO₃H radical,
Z′ represents an ion of an alkaline or alkaline earth metal such as sodium, an ammonium ion or an ion issued from an organic amine, and
R₁′ denotes an alkyl radical of an acid R₁′—COOH present in coconut oil or in hydrolysed linseed oil, an alkyl radical, such as a C₇, C₉, C₁₁ or C₁₃ alkyl radical, a C₁₇ alkyl radical and its iso form, or an unsaturated C₁₇ radical.

It is preferable that the amphoteric surfactant be selected from (C₈-C₂₄) alkyl amphomonoacetates, (C₈-C₂₄) alkyl amphodiacetates, (C₈-C₂₄) alkyl amphomonopropionates, and (C₈-C₂₄) alkyl amphodipropionates.

These compounds are classified in the CTFA dictionary, 5th edition, 1993, under the names Disodium Cocoamphodiacetate, Disodium Lauroamphodiacetate, Disodium Caprylamphodiacetate, Disodium Capryloamphodiacetate, Disodium Cocoamphodipropionate, Disodium Lauroamphopropionate, Disodium Caprylamphodipropionate, Disodium Caprylamphodipropionate, Lauroamphodipropionic acid and Cocoamphodipropionic acid.

By way of example, mention may be made of the cocoamphodiacetate sold under the trade name Miranol® C2M concentrate by the company Rhodia Chimie.

### (f3) Cationic Surfactants

According to the present invention, the type of cationic surfactant is not limited. The cationic surfactant may be selected from the group consisting of optionally polyoxyalkylenated, primary, secondary or tertiary fatty amine salts, quaternary ammonium salts, and mixtures thereof.

Examples of quaternary ammonium salts that may be mentioned include, but are not limited to: those of general formula (I) below:

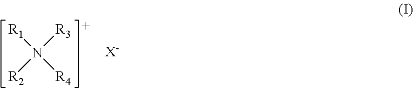

wherein
R₁, R₂, R₃, and R₄, which may be identical or different, are chosen from linear and branched aliphatic radicals comprising from 1 to 30 carbon atoms and optionally comprising heteroatoms such as oxygen, nitrogen, sulfur and halogens. The aliphatic radicals may be chosen, for example, from alkyl, alkoxy, C₂-C₆ polyoxyalkylene, alkylamide, (C₁₂-C₂₂) alkylamido (C₂-C₆) alkyl, (C₁₂-C₂₂) alkylacetate and hydroxyalkyl radicals; and aromatic radicals such as aryl and alkylaryl; and X⁻ is chosen from halides, phosphates, acetates, lactates, (C₂-C₆) alkyl sulfates and alkyl- or alkylaryl-sulfonates;
quaternary ammonium salts of imidazoline, for instance those of formula (II) below:

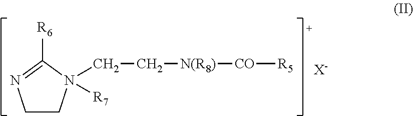

wherein:
R₅ is chosen from alkenyl and alkyl radicals comprising from 8 to 30 carbon atoms, for example fatty acid derivatives of tallow or of coconut;
R₆ is chosen from hydrogen, C₁-C₄ alkyl radicals, and alkenyl and alkyl radicals comprising from 8 to 30 carbon atoms;
R₇ is chosen from C₁-C₄ alkyl radicals;
R₈ is chosen from hydrogen and C₁-C₄ alkyl radicals; and
X⁻ is chosen from halides, phosphates, acetates, lactates, alkyl sulfates, alkyl sulfonates, and alkylaryl sulfonates. In one embodiment, R₅ and R₆ are, for example, a mixture of radicals chosen from alkenyl and alkyl radicals comprising from 12 to 21 carbon atoms, such as fatty acid derivatives of tallow, R₇ is methyl and R₈ is hydrogen. Examples of such products include, but are not limited to, Quaternium-27 (CTFA 1997) and Quaternium-83 (CTFA 1997), which are sold under the names “Rewoquat®” W75, W90, W75PG and W75HPG by the company Witco; diquaternary ammonium salts of formula (III):

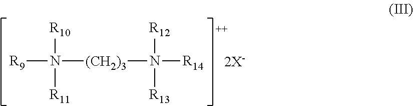

wherein:
R₉ is chosen from aliphatic radicals comprising from 16 to 30 carbon atoms;
R₁₀ is chosen from hydrogen or alkyl radicals comprising from 1 to 4 carbon atoms or a group (R₁₆ₐ)(R₁₇ₐ)(R₁₈)N⁺(CH₂)₃;
R₁₁, R₁₂, R₁₃, R₁₄, R₁₆ₐ, R₁₇ₐ, and R₁₈ₐ, which may be identical or different, are chosen from hydrogen and alkyl radicals comprising from 1 to 4 carbon atoms; and
X⁻ is chosen from halides, acetates, phosphates, nitrates, ethyl sulfates, and methyl sulfates.

An example of one such diquaternary ammonium salt is FINQUAT CTP of FINETEX (Quaternium-89) or FINQUAT CT of FINETEX (Quaternium-75); and quaternary ammonium salts comprising at least one ester function, such as those of formula (IV) below:

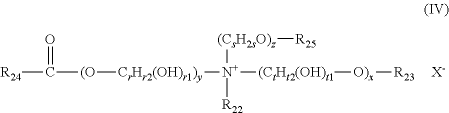

wherein:
R₂₂ is chosen from C₁-C₆ alkyl radicals and C₁-C₆ hydroxyalkyl and dihydroxyalkyl radicals;
R₂₃ is chosen from:
the radical below:

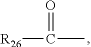

linear and branched, saturated and unsaturated C₁-C₂₂ hydrocarbon-based radicals R₂₇, and hydrogen,
R₂₅ is chosen from:
the radical below:

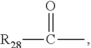

linear and branched, saturated and unsaturated C₁-C₆ hydrocarbon-based radicals R₂₉, and hydrogen,
R₂₄, R₂₆, and R₂₈, which may be identical or different, are chosen from linear and branched, saturated and unsaturated, C₇-C₂₁, hydrocarbon-based radicals;
r, s, and t, which may be identical or different, are chosen from integers ranging from 2 to 6;
each of r1 and t1, which may be identical or different, is 0 or 1, and r2+r1=2r and t1+2t=2t;
y is chosen from integers ranging from 1 to 10;
x and z, which may be identical or different, are chosen from integers ranging from 0 to 10;

X⁻ is chosen from simple and complex, organic and inorganic anions; with the proviso that the sum x+y+z ranges from 1 to 15, that when x is 0, R₂₃ denotes R₂₇, and that when z is 0, R₂₅ denotes R₂₉. R₂₂ may be chosen from linear and branched alkyl radicals. In one embodiment, R₂₂ is chosen from linear alkyl radicals. In another embodiment, R₂₂ is chosen from methyl, ethyl, hydroxyethyl, and dihydroxypropyl radicals, for example methyl and ethyl radicals. In one embodiment, the sum x+y+z ranges from 1 to 10. When R₂₃ is a hydrocarbon-based radical R₂₇, it may be long and comprise from 12 to 22 carbon atoms, or short and comprise from 1 to 3 carbon atoms. When R₂₅ is a hydrocarbon-based radical R₂₉, it may comprise, for example, from 1 to 3 carbon atoms. By way of a non-limiting example, in one embodiment, R₂₄, R₂₆, and R₂₈, which may be identical or different, are chosen from linear and branched, saturated and unsaturated, C₁₁-C₂₁ hydrocarbon-based radicals, for example from linear and branched, saturated and unsaturated C₁₁-C₂₁ alkyl and alkenyl radicals. In another embodiment, x and z, which may be identical or different, are 0 or 1. In one embodiment, y is equal to 1. In another embodiment, r, s and t, which may be identical or different, are equal to 2 or 3, for example equal to 2. The anion X⁻ may be chosen from, for example, halides, such as chloride, bromide, and iodide; and C₁-C₄ alkyl sulfates, such as methyl sulfate. However, methanesulfonate, phosphate, nitrate, tosylate, an anion derived from an organic acid, such as acetate and lactate, and any other anion that is compatible with the ammonium comprising an ester function, are other non-limiting examples of anions that may be used according to the present invention. In one embodiment, the anion X⁻ is chosen from chloride and methyl sulfate.

In another embodiment, the ammonium salts of formula (IV) may be used, wherein:

R₂₂ is chosen from methyl and ethyl radicals,
x and y are equal to 1;
z is equal to 0 or 1;
r, s and t are equal to 2;
R₂₃ is chosen from:
the radical below:

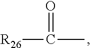

methyl, ethyl, and C₁₄-C₂₂ hydrocarbon-based radicals, and hydrogen;
R₂₅ is chosen from:
the radical below:

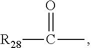

and hydrogen;

R₂₄, R₂₆, and R₂₈, which may be identical or different, are chosen from linear and branched, saturated and unsaturated, C₁₃-C₁₇ hydrocarbon-based radicals, for example from linear and branched, saturated and unsaturated, C₁₃-C₁₇ alkyl and alkenyl radicals.

In one embodiment, the hydrocarbon-based radicals are linear.

Non-limiting examples of compounds of formula (IV) that may be mentioned include salts, for example chloride and methyl sulfate, of diacyloxyethyl-dimethylammonium, of diacyloxyethyl-hydroxyethyl-methylammonium, of monoacyloxyethyl-dihydroxyethyl-methylammonium, of triacyloxyethyl-methylammonium, of monoacyloxyethyl-hydroxyethyl-dimethylammonium, and mixtures thereof. In one embodiment, the acyl radicals may comprise from 14 to 18 carbon atoms, and may be derived, for example, from a plant oil, for instance palm oil and sunflower oil. When the compound comprises several acyl radicals, these radicals may be identical or different.

These products may be obtained, for example, by direct esterification of optionally oxyalkylenated triethanolamine, triisopropanolamine, alkyldiethanolamine or alkyldiisopropanolamine onto fatty acids or onto mixtures of fatty acids of plant or animal origin, or by transesterification of the methyl esters thereof. This esterification may be followed by a quaternization using an alkylating agent chosen from alkyl halides, for example methyl and ethyl halides; dialkyl sulfates, for example dimethyl and diethyl sulfates; methyl methanesulfonate; methyl para-toluenesulfonate; glycol chlorohydrin; and glycerol chlorohydrin.

Such compounds are sold, for example, under the names Dehyquart® by the company Cognis, Stepanquat® by the company Stepan, Noxamium® by the company Ceca, and “Rewoquat® WE 18” by the company Rewo-Goldschmidt.

Other non-limiting examples of ammonium salts that may be used in the compositions according to the present invention include the ammonium salts comprising at least one ester function described in U.S. Pat. Nos. 4,874,554 and 4,137,180.

The quaternary ammonium salts mentioned above that may be used in compositions according to the present invention include, but are not limited to, those corresponding to formula (I), for example tetraalkylammonium chlorides, for instance dialkyldimethylammonium and alkyltrimethylammonium chlorides in which the alkyl radical comprises from about 12 to 22 carbon atoms, such as behenyltrimethylammonium, distearyldimethylammonium, cetyltrimethylammonium and benzyldimethylstearylammonium chloride; palmitylamidopropyltrimethylammonium chloride; and stearamidopropyldimethyl(myristyl acetate)ammonium chloride, sold under the name “Ceraphyl® 70” by the company Van Dyk.

According to one embodiment, the cationic surfactant that may be used in the compositions of the present invention is chosen from quaternary ammonium salts, for example from behenyltrimethylammonium chloride, cetyltrimethylammonium chloride, Quaternium-83, Quaternium-87, Quaternium-22, behenylamidopropyl-2,3-dihydroxypropyldimethylammonium chloride, palmitylamidopropyltrimethylammonium chloride, and stearamidopropyldimethylamine.

### (f4) Nonionic Surfactants

The nonionic surfactants are compounds well known in themselves (see, e.g., in this regard, “Handbook of Surfactants” by M. R. Porter, Blackie & Son publishers (Glasgow and London), 1991, pp. 116-178). Thus, they can, for example, be chosen from alcohols, alpha-diols, alkylphenols and esters of fatty acids, these compounds being ethoxylated, propoxylated or glycerolated and having at least one fatty chain comprising, for example, from 8 to 30 carbon atoms, it being possible for the number of ethylene oxide or propylene oxide groups to range from 2 to 50, and for the number of glycerol groups to range from 1 to 30. Maltose derivatives may also be mentioned. Non-limiting mention may also be made of copolymers of ethylene oxide and/or of propylene oxide; condensates of ethylene oxide and/or of propylene oxide with fatty alcohols; polyethoxylated fatty amides comprising, for example, from 2 to 30 mol of ethylene oxide; polyglycerolated fatty amides comprising, for example, from 1.5 to 5 glycerol groups, such as from 1.5 to 4; ethoxylated fatty acid esters of sorbitan comprising from 2 to 30 mol of ethylene oxide; ethoxylated oils of plant origin; fatty acid esters of sucrose; fatty acid esters of polyethylene glycol; polyethoxylated fatty acid mono or diesters of glycerol (C₆-C₂₄)alkylpolyglycosides; N—(C₆-C₂₄)alkylglucamine derivatives; amine oxides such as (C₁₀-C₁₄)alkylamine oxides or N—(C₁₀-C₁₄)acylaminopropylmorpholine oxides; silicone surfactants; and mixtures thereof.

The nonionic surfactants may preferably be chosen from monooxyalkylenated, polyoxyalkylenated, monoglycerolated or polyglycerolated nonionic surfactants. The oxyalkylene units are more particularly oxyethylene or oxypropylene units, or a combination thereof, and are preferably oxyethylene units.

Examples of monooxyalkylenated or polyoxyalkylenated nonionic surfactants that may be mentioned include:

monooxyalkylenated or polyoxyalkylenated (C₈-C₂₄)alkylphenols, saturated or unsaturated, linear or branched, monooxyalkylenated or polyoxyalkylenated C₈-C₃₀ alcohols,
saturated or unsaturated, linear or branched, monooxyalkylenated or polyoxyalkylenated C₈-C₃₀ amides,
esters of saturated or unsaturated, linear or branched, C₈-C₃₀ acids and of polyalkylene glycols, monooxyalkylenated or polyoxyalkylenated esters of saturated or unsaturated, linear or branched, C₈-C₃₀ acids and of sorbitol,
saturated or unsaturated, monooxyalkylenated or polyoxyalkylenated plant oils, condensates of ethylene oxide and/or of propylene oxide, inter alia, alone or as mixtures.

The surfactants preferably contain a number of moles of ethylene oxide and/or of propylene oxide of between 1 and 100 and most preferably between 2 and 50. Advantageously, the nonionic surfactants do not comprise any oxypropylene units.

According to one of the embodiments of the present invention, the polyoxyalkylenated nonionic surfactants are chosen from polyoxyethylenated fatty alcohol (polyethylene glycol ether of fatty alcohol) and polyoxyethylenated fatty ester (polyethylene glycol ester of fatty acid).

Examples of polyoxyethylenated fatty alcohol (or C₈-C₃₀ alcohols) that may be mentioned include the adducts of ethylene oxide with lauryl alcohol, especially those containing from 9 to 50 oxyethylene units and more particularly those containing from 10 to 12 oxyethylene units (Laureth-10 to Laureth-12, as the CTFA names); the adducts of ethylene oxide with behenyl alcohol, especially those containing from 9 to 50 oxyethylene units (Beheneth-9 to Beheneth-50, as the CTFA names); the adducts of ethylene oxide with cetearyl alcohol (mixture of cetyl alcohol and stearyl alcohol), especially those containing from 10 to 30 oxyethylene units (Ceteareth-10 to Ceteareth-30, as the CTFA names); the adducts of ethylene oxide with cetyl alcohol, especially those containing from 10 to 30 oxyethylene units (Ceteth-10 to Ceteth-30, as the CTFA names);

the adducts of ethylene oxide with stearyl alcohol, especially those containing from 10 to 30 oxyethylene units (Steareth-10 to Steareth-30, as the CTFA names); the adducts of ethylene oxide with isostearyl alcohol, especially those containing from 10 to 50 oxyethylene units (Isosteareth-10 to Isosteareth-50, as the CTFA names); and mixtures thereof

As examples of monoglycerolated or polyglycerolated nonionic surfactants, monoglycerolated or polyglycerolated C₈-C₄₀ alcohols are preferably used.

In particular, the monoglycerolated or polyglycerolated C₈-C₄₀ alcohols correspond to the following formula:

RO—[CH₂—CH(CH₂OH)—O]ₘ—H or RO—[CH(CH₂OH)—CH₂O]ₘ—H

in which R represents a linear or branched C₈-C₄₀ and preferably C₈-C₃₀ alkyl or alkenyl radical, and m represents a number ranging from 1 to 30 and preferably from 1.5 to 10.

As examples of compounds that are suitable in the context of the present invention, mention may be made of lauryl alcohol containing 4 mol of glycerol (INC′ name: Polyglyceryl-4 Lauryl Ether), lauryl alcohol containing 1.5 mol of glycerol, oleyl alcohol containing 4 mol of glycerol (INC′ name: Polyglyceryl-4 Oleyl Ether), oleyl alcohol containing 2 mol of glycerol (INCI name: Polyglyceryl-2 Oleyl Ether), cetearyl alcohol containing 2 mol of glycerol, cetearyl alcohol containing 6 mol of glycerol, oleocetyl alcohol containing 6 mol of glycerol, and octadecanol containing 6 mol of glycerol.

The alcohol may represent a mixture of alcohols in the same way that the value of m represents a statistical value, which means that, in a commercial product, several species of polyglycerolated fatty alcohol may coexist in the form of a mixture.

Among the monoglycerolated or polyglycerolated alcohols, it is preferable to use the C₈/C₁₀ alcohol containing 1 mol of glycerol, the C₁₀/C₁₂, alcohol containing 1 mol of glycerol and the C₁₂ alcohol containing 1.5 mol of glycerol.

Examples of polyoxyethylenated fatty esters that may be mentioned include the adducts of ethylene oxide with esters of lauric acid, palmitic acid, stearic acid or behenic acid, and mixtures thereof, especially those containing from 9 to 100 oxyethylene units, such as PEG-9 to PEG-50 laurate (CTFA names: PEG-9 laurate to PEG-50 laurate); PEG-9 to PEG-50 palmitate (CTFA names: PEG-9 palmitate to PEG-50 palmitate); PEG-9 to PEG-50 stearate (CTFA names: PEG-9 stearate to PEG-50 stearate); PEG-9 to PEG-50 palmitostearate; PEG-9 to PEG-50 behenate (CTFA names: PEG-9 behenate to PEG-50 behenate); polyethylene glycol 100 EO monostearate (CTFA name: PEG-100 stearate); and mixtures thereof.

According to one of the embodiments of the present invention, the nonionic surfactant may be selected from esters of polyols with fatty acids with a saturated or unsaturated chain containing for example from 8 to 24 carbon atoms, preferably 12 to 22 carbon atoms, and polyoxyalkylenated derivatives thereof, preferably containing from 10 to 200, and more preferably from 10 to 100 oxyalkylene units, such as glyceryl esters of a C₈-C₂₄, preferably C₁₂-C₂₂, fatty acid or acids and polyoxyalkylenated derivatives thereof, preferably containing from 10 to 200, and more preferably from 10 to 100 oxyalkylene units; sorbitol esters of a C₈-C₂₄, preferably C₁₂-C₂₂, fatty acid or acids and polyoxyalkylenated derivatives thereof, preferably containing from 10 to 200, and more preferably from 10 to 100 oxyalkylene units; sugar (sucrose, maltose, glucose, fructose, and/or alkylglycose) esters of a C₈-C₂₄, preferably C₁₂-C₂₂, fatty acid or acids and polyoxyalkylenated derivatives thereof, preferably containing from 10 to 200, and more preferably from 10 to 100 oxyalkylene units; ethers of fatty alcohols; ethers of sugar and a C₈-C₂₄, preferably C₁₂-C₂₂, fatty alcohol or alcohols; and mixtures thereof.

As glyceryl esters of fatty acids, glyceryl stearate (glyceryl mono-, di- and/or tristearate) (CTFA name: glyceryl stearate) or glyceryl ricinoleate and mixtures thereof can be cited, and as polyoxyalkylenated derivatives thereof, mono-, di- or triester of fatty acids with a polyoxyalkylenated glycerol (mono-, di- or triester of fatty acids with a polyalkylene glycol ether of glycerol), preferably polyoxyethylenated glyceryl stearate (mono-, di- and/or tristearate), such as PEG-20 glyceryl stearate (mono-, di- and/or tristearate) can be cited.

Mixtures of these surfactants, such as for example the product containing glyceryl stearate and PEG-100 stearate, marketed under the name ARLACEL 165 by Uniqema, and the product containing glyceryl stearate (glyceryl mono- and distearate) and potassium stearate marketed under the name TEGIN by Goldschmidt (CTFA name: glyceryl stearate SE), can also be used.

The sorbitol esters of C₈-C₂₄ fatty acids and polyoxyalkylenated derivatives thereof can be selected from sorbitan palmitate, sorbitan isostearate, sorbitan trioleate and esters of fatty acids and alkoxylated sorbitan containing for example from 20 to 100 EO, such as for example sorbitan monostearate (CTFA name: sorbitan stearate), sold by the company ICI under the name Span 60, sorbitan monopalmitate (CTFA name: sorbitan palmitate), sold by the company ICI under the name Span 40, and sorbitan tristearate 20 EO (CTFA name: polysorbate 65), sold by the company ICI under the name Tween 65, polyethylene sorbitan trioleate (polysorbate 85) or the compounds marketed under the trade names Tween 20 or Tween 60 by Uniqema.

As esters of fatty acids and glucose or alkylglucose, glucose palmitate, alkylglucose sesquistearates such as methylglucose sesquistearate, alkylglucose palmitates such as methylglucose or ethylglucose palmitate, methylglucoside fatty esters, the diester of methylglucoside and oleic acid (CTFA name: Methyl glucose dioleate), the mixed ester of methylglucoside and the mixture of oleic acid/hydroxystearic acid (CTFA name: Methyl glucose dioleate/hydroxystearate), the ester of methylglucoside and isostearic acid (CTFA name: Methyl glucose isostearate), the ester of methylglucoside and lauric acid (CTFA name: Methyl glucose laurate), the mixture of monoester and diester of methylglucoside and isostearic acid (CTFA name: Methyl glucose sesqui-isostearate), the mixture of monoester and diester of methylglucoside and stearic acid (CTFA name: Methyl glucose sesquistearate) and in particular the product marketed under the name Glucate SS by AMERCHOL, and mixtures thereof can be cited.

As ethoxylated ethers of fatty acids and glucose or alkylglucose, ethoxylated ethers of fatty acids and methylglucose, and in particular the polyethylene glycol ether of the diester of methylglucose and stearic acid with about 20 moles of ethylene oxide (CTFA name: PEG-20 methyl glucose distearate) such as the product marketed under the name Glucam E-20 distearate by AMERCHOL, the polyethylene glycol ether of the mixture of monoester and diester of methyl-glucose and stearic acid with about 20 moles of ethylene oxide (CTFA name: PEG-20 methyl glucose sesquistearate) and in particular the product marketed under the name Glucamate SSE-20 by AMERCHOL and that marketed under the name Grillocose PSE-20 by GOLDSCHMIDT, and mixtures thereof, can for example be cited.

As sucrose esters, saccharose palmito-stearate, saccharose stearate and saccharose monolaurate can for example be cited.

As sugar ethers, alkylpolyglucosides can be used, and for example decylglucoside such as the product marketed under the name MYDOL 10 by Kao Chemicals, the product marketed under the name PLANTAREN 2000 by Henkel, and the product marketed under the name ORAMIX NS 10 by Seppic, caprylyl/capryl glucoside such as the product marketed under the name ORAMIX CG 110 by Seppic or under the name LUTENSOL GD 70 by BASF, laurylglucoside such as the products marketed under the names PLANTAREN 1200 N and PLANTACARE 1200 by Henkel, coco-glucoside such as the product marketed under the name PLANTACARE 818/UP by Henkel, cetostearyl glucoside possibly mixed with cetostearyl alcohol, marketed for example under the name MONTANOV 68 by Seppic, under the name TEGO-CARE CG90 by Goldschmidt and under the name EMULGADE KE3302 by Henkel, arachidyl glucoside, for example in the form of the mixture of arachidyl and behenyl alcohols and arachidyl glucoside marketed under the name MONTANOV 202 by Seppic, cocoylethylglucoside, for example in the form of the mixture (35/65) with cetyl and stearyl alcohols, marketed under the name MONTANOV 82 by Seppic, and mixtures thereof can in particular be cited.

Mixtures of glycerides of alkoxylated plant oils such as mixtures of ethoxylated (200 EO) palm and copra (7 EO) glycerides can also be cited.

The nonionic surfactant according to the present invention preferably contains alkenyl or branched C₁₂-C₂₂ acyl chain such as oleyl or isostearyl group. More preferably, the nonionic surfactant according to the present invention is PEG-20 glyceryl triisostearate.

According to one of the embodiments of the present invention, the nonionic surfactant may be selected from polyoxyethylenated (1-40 EO) and polyoxypropylenated (1-30 PO) alkyl (C₁₆-C₃₀) ethers.

The polyoxyethylenated (1-40 EO) and polyoxypropylenated (1-30 PO) alkyl (C₁₆-C₃₀) ethers which may be used as surfactants in the nanoemulsion according to the present invention, may be selected from the group consisting of:

PPG-6 Decyltetradeceth-30; Polyoxyethlene (30) Polyoxypropylene (6) Tetradecyl Ether such as those sold as Nikkol PEN-4630 from Nikko Chemicals Co.,
PPG-6 Decyltetradeceth-12; Polyoxyethylene (12) Polyoxypropylene (6) Tetradecyl Ether such as those sold as Nikkol PEN-4612 from Nikko Chemicals Co.,
PPG-13 Decyltetradeceth-24; Polyoxyethylene (24) Polyoxypropylene (13) Decyltetradecyl Ether such as those sold as UNILUBE 50MT-2200B from NOF Corporation,
PPG-6 Decyltetradeceth-20; Polyoxyethylene (20) Polyoxypropylene (6) Decyltetradecyl Ether such as those sold as Nikkol PEN-4620 from Nikko Chemicals Co.,
PPG-4 Ceteth-1; Polyoxyethylene (1) Polyoxypropylene (4) Cetyl Ether such as those sold as Nikkol PBC-31 from Nikko Chemicals Co.,
PPG-8 Ceteth-1; Polyoxyethylene (1) Polyoxypropylene (8) Cetyl Ether such as those sold as Nikkol PBC-41 from Nikko Chemicals Co.,
PPG-4 Ceteth-10; Polyoxyethylene (10) Polyoxypropylene (4) Cetyl Ether such as those sold as Nikkol PBC-33 from Nikko Chemicals Co.,
PPG-4 Ceteth-20; Polyoxyethylene (20) Polyoxypropylene (4) Cetyl Ether such as those sold as Nikkol PBC-34 from Nikko Chemicals Co.,
PPG-5 Ceteth-20; Polyoxyethylene (20) Polyoxypropylene (5) Cetyl Ether such as those sold as Procetyl AWS from Croda Inc.,
PPG-8 Ceteth-20; Polyoxyethylene (20) Polyoxypropylene (8) Cetyl Ether such as those sold as Nikko′ PBC-44 from Nikko Chemicals Co., and
PPG-23 Steareth-34; Polyoxyethylene Polyoxypropylene Stearyl Ether (34 EO) (23 PO) such as those sold as Unisafe 34S-23 from Pola Chemical Industries. They can provide a composition with stability for a long time, even though the temperature of the composition is increased and decreased in a relatively short period of time.

It is more preferable that the polyoxyethylenated (1-40 EO) and polyoxypropylenated (1-30 PO) alkyl (C₁₆-C₃₀) ethers are (15-40 EO) and polyoxypropylenated (5-30 PO) alkyl (C₁₆-C₂₄) ethers, which could be selected from the group consisting of PPG-6 Decyltetradeceth-30, PPG-13 Decyltetradeceth-24, PPG-6 Decyltetradeceth-20, PPG-5 Ceteth-20, PPG-8 Ceteth-20, and PPG-23 Steareth-34.

It is most preferable that the polyoxyethylenated (1-40 EO) and polyoxypropylenated (1-30 PO) alkyl (C₁₆-C₃₀) ethers are (15-40 EO) and polyoxypropylenated (5-30 PO) alkyl (C₁₆-C₂₄) ethers, which could be selected from the group consisting of PPG-6 Decyltetradeceth-30, PPG-13 Decyltetradeceth-24, PPG-5 Ceteth-20, and PPG-8 Ceteth-20. They can also provide a composition with transparency for a long time.

According to one of the embodiments of the present invention, the nonionic surfactant may be selected from copolymers of ethylene oxide and of propylene oxide, in particular copolymers of the following formula:

HO(C₂H₄O)ₐ(C₃H₆O)_{b}(C₂H₄O)_{c}H

in which a, b and c are integers such that a+c ranges from 2 to 100 and b ranges from 14 to 60, and mixtures thereof.

According to one of the embodiments of the present invention, the nonionic surfactant may be selected from silicone surfactants. Non-limiting mention may be made of those disclosed in documents U.S. Pat. No. 5,364,633 and U.S. Pat. No. 5,411,744.

The silicone surfactant may preferably be a compound of formula (I):

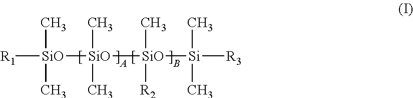

in which:
R₁, R₂ and R₃, independently of each other, represent a C₁-C₆ alkyl radical or a radical —(CH₂)ₓ—(OCH₂CH₂)_{y}—(OCH₂CH₂CH₂)_{z}—OR₄, at least one radical R₁, R₂ or R₃ not being an alkyl radical; R₄ being a hydrogen, an alkyl radical or an acyl radical;
A is an integer ranging from 0 to 200;
B is an integer ranging from 0 to 50; with the proviso that A and B are not simultaneously equal to zero;
x is an integer ranging from 1 to 6;
y is an integer ranging from 1 to 30;
z is an integer ranging from 0 to 5.

According to one preferred embodiment of the present invention, in the compound of formula (I), the alkyl radical is a methyl radical, x is an integer ranging from 2 to 6 and y is an integer ranging from 4 to 30.

As examples of silicone surfactants of formula (I), mention may be made of the compounds of formula (II):

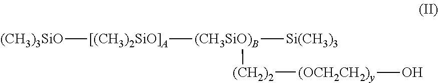

in which A is an integer ranging from 20 to 105, B is an integer ranging from 2 to 10 and y is an integer ranging from 10 to 20.

As examples of silicone surfactants of formula (I), mention may also be made of the compounds of formula (III):

H—(OCH₂CH₂)_{y}—(CH₂)₃—[(CH₃)₂SiO]_{A′}—(CH₂)₃—(OCH₂CH₂)_{y}—OH (III)

in which A′ and y are integers ranging from 10 to 20.

Compounds of the present invention which may be used are those sold by the company Dow Corning under the names DC 5329, DC 7439-146, DC 2-5695 and Q4-3667. The compounds DC 5329, DC 7439-146 and DC 2-5695 are compounds of formula (II) in which, respectively, A is 22, B is 2 and y is 12; A is 103, B is 10 and y is 12; A is 27, B is 3 and y is 12.

The compound Q4-3667 is a compound of formula (III) in which A is 15 and y is 13.

The amount of the (f) additional surfactant different from the (b) polyglyceryl fatty acid ester is not limited, and may range from 0.01 to 10% by weight, preferably from 0.05 to 5% by weight, and more preferably from 0.1 to 2% by weight, relative to the total weight of the composition.

### [Associative Polymer]

The composition according to the present invention may further comprise at least one associative polymer. A single type of associative polymer may be used, but two or more different types of associative polymer may be used in combination.

Associative polymers are water-soluble polymers that are capable, in an aqueous medium, of reversibly combining with each other or with other molecules.

Their chemical structure comprises one or more hydrophilic regions and one or more hydrophobic regions characterized by the presence of at least one C₈-C₃₀ fatty chain.

Among the (g) associative polymers of anionic type that may be mentioned are:
(I) those comprising at least one hydrophilic unit and at least one fatty-chain allyl ether unit, more particularly those whose hydrophilic unit consists of an ethylenic unsaturated anionic monomer, more particularly of a vinylcarboxylic acid, and most particularly of an acrylic acid or a methacrylic acid, or mixtures thereof, the fatty-chain allyl ether unit thereof corresponds to the monomer of formula (XXI) below:

CH₂═CR′CH₂OBₙR (XXI)

in which R′ denotes H or CH₃, B denotes an ethylenoxy radical, n is zero or denotes an integer ranging from 1 to 100, and R denotes a hydrocarbon-based radical chosen from alkyl, arylalkyl, aryl, alkylaryl, and cycloalkyl radicals, comprising from 8 to 30 carbon atoms, preferably 10 to 24, and even more particularly from 12 to 18 carbon atoms. The unit of formula (XXI) that is more particularly preferred is a unit in which R′ denotes H, n is equal to 10, and R denotes a stearyl radical (C₁₈).

Anionic associative polymers of this type are described and prepared, according to an emulsion polymerization process, in patent EP-0 216 479.

Among these anionic associative polymers that are particularly preferred according to the present invention are polymers formed from 20% to 60% by weight of acrylic acid and/or of methacrylic acid, from 5% to 60% by weight of lower alkyl (meth)acrylates, from 2% to 50% by weight of the fatty-chain allyl ether of formula (XXI), and from 0 to 1% by weight of a crosslinking agent which is a well-known copolymerizable unsaturated polyethylenic monomer, for instance diallyl phthalate, allyl (meth)acrylate, divinylbenzene, (poly)ethylene glycol dimethacrylate, or methylenebisacrylamide.

Among the latter polymers, those most particularly preferred are crosslinked terpolymers of methacrylic acid, of ethyl acrylate and of polyethylene glycol (10 EO) stearyl alcohol ether (Steareth-10), in particular those sold by the company Allied Colloids under the names Salcare SC 80® and Salcare SC 90®, which are aqueous 30% emulsions of a crosslinked terpolymer of methacrylic acid, of ethyl acrylate, and of steareth-10 allyl ether (40/50/10).
(II) polymers comprising at least one hydrophilic unit of unsaturated olefinic carboxylic acid type, and at least one hydrophobic unit of a type such as a (C₁₀-C₃₀) alkyl ester of an unsaturated carboxylic acid.

These polymers are preferably chosen from those in which the hydrophilic unit of the unsaturated olefinic carboxylic acid type corresponds to the monomer of formula (XXII) below:

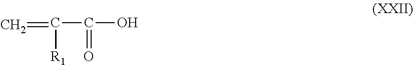

in which R₁ denotes H, or CH₃ or C₂H₅, i.e., acrylic acid, methacrylic acid, or ethacrylic acid units, and whose hydrophobic unit of a type such as a (C₁₀-C₃₀) alkyl ester of an unsaturated carboxylic acid corresponds to the monomer of formula (XXIII) below:

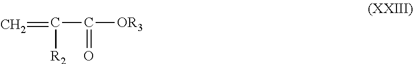

in which R₂ denotes H, or CH₃ or C₂H₅ (i.e., acrylate, methacrylate, or ethacrylate units) and preferably H (acrylate units) or CH₃ (methacrylate units), and R₃ denotes a C₁₀-C₃₀ and preferably C₁₂-C₂₂ alkyl radical.

(C₁₀-C₃₀) alkyl esters of unsaturated carboxylic acids in accordance with the present invention comprise, for example, lauryl acrylate, stearyl acrylate, decyl acrylate, isodecyl acrylate, and dodecyl acrylate, and the corresponding methacrylates, lauryl methacrylate, stearyl methacrylate, decyl methacrylate, isodecyl methacrylate, and dodecyl methacrylate.

Anionic polymers of this type are described and prepared, for example, according to patents U.S. Pat. No. 3,915,921 and U.S. Pat. No. 4,509,949.

According to a preferred embodiment, these polymers are crosslinked.

Among the anionic associative polymers of this type that will be used more particularly are polymers formed from a monomer mixture comprising:

(i) essentially acrylic acid,
(ii) an ester of formula (XXIII) described above and in which R₂ denotes H or CH₃, and R₃ denotes an alkyl radical containing from 12 to 22 carbon atoms, and
(iii) a crosslinking agent, which is a well-known copolymerizable unsaturated polyethylenic monomer, for instance diallyl phthalate, allyl (meth)acrylate, divinylbenzene, (poly)ethylene glycol dimethacrylate, and methylenebisacrylamide.

Among the said above polymers, those most particularly preferred according to the present invention are acrylate/C₁₀-C₃₀ alkyl acrylate copolymers (INCI name: Acrylates/C10-30 Alkyl Acrylate Crosspolymer), such as Pemulen TR1®, Pemulen TR2®, Carbopol 1382®, and Carbopol EDT2020® from Lubrizol, and TEGO® Carbomer 841 SER from Evonik, and even more preferentially TEGO® Carbomer 841 SER.
(III) maleic anhydride/C₃₀-C₃₈α-olefin/alkyl maleate terpolymers, such as the product (maleic anhydride/C₃₀-C₃₈ α-olefin/isopropyl maleate) sold under the name Performa V 1608® by the company New Phase Technologies.
(IV) acrylic terpolymers comprising:
(a) about 20% to 70% by weight of a carboxylic acid containing α,β-monoethylenic unsaturation,
(b) about 20% to 80% by weight of a non-surfactant monomer containing an α,β-monoethylenic unsaturation other than (a), and
(c) about 0.5% to 60% by weight of a nonionic monourethane which is the product of a reaction of a monohydric surfactant with a monoisocyanate containing a monoethylenic unsaturation, such as those described in patent application EP-A-0 173 109 and more particularly the terpolymer described in Example 3, namely a methacrylic acid/methyl acrylate/behenyl alcohol dimethyl-meta-isopropenylbenzylisocyanate ethoxylated (40 EO) terpolymer, as an aqueous 25% dispersion.
   (V) copolymers comprising among their monomers a carboxylic acid containing an α,β-monoethylenic unsaturation and an ester of a carboxylic acid containing an α,β-monoethylenic unsaturation and of an oxyalkylenated fatty alcohol.

Preferentially, these compounds also comprise as a monomer an ester of an α,β-monoethylenically unsaturated carboxylic acid and of a C₁-C₄ alcohol.

An example of a compound of this type that may be mentioned is Aculyn 22® sold by the company Rohm & Haas, which is a methacrylic acid/ethyl acrylate/oxyalkylenated stearyl methacrylate terpolymer.

Among the associative polymers of cationic type that may be mentioned are:
(I) cationic associative polyurethanes, the family of which has been described by the Applicant in French patent application No. 00/09609; these may be represented by the general formula (XXIV) below:

R—X—(P)n-[L-(Y)m]r-L′-(P′)p-X′—R′ (XXIV)

in which:
R and R′, which may be identical or different, represent a hydrophobic group or a hydrogen atom;
X and X′, which may be identical or different, represent a group comprising an amine function optionally bearing a hydrophobic group, or alternatively a group L″;
L, L′, and L″, which may be identical or different, represent a group derived from a diisocyanate;
P and P′, which may be identical or different, represent a group comprising an amine function optionally bearing a hydrophobic group;
Y represents a hydrophilic group;
r is an integer between 1 and 100, preferably between 1 and 50, and in particular between 1 and 25;
n, m, and p each range, independently of each other, from 0 to 1000; and
the molecule contains at least one protonated or quaternized amine function and at least one hydrophobic group.

In one preferred embodiment of these polyurethanes, the only hydrophobic groups are the groups R and R′ at the chain ends.

One preferred family of cationic associative polyurethanes is the one corresponding to formula (XXIV) described above and in which:

R and R′ both independently represent a hydrophobic group,
X and X′ each represent a group L″,
n and p are between 1 and 1000, and
L, L′, L″, P, P′, Y, and m have the meanings given above.

Another preferred family of cationic associative polyurethanes is the one corresponding to formula (XXIV) above in which:

R and R′ both independently represent a hydrophobic group, X and X′ each represent a group L″, n and p are 0, and L, L′, L″, Y, and m have the meanings given above.

The fact that n and p are 0 means that these polymers do not comprise units derived from a monomer containing an amine function, incorporated into the polymer during the polycondensation. The protonated amine functions of these polyurethanes result from the hydrolysis of excess isocyanate functions, at the chain end, followed by alkylation of the primary amine functions formed with alkylating agents containing a hydrophobic group, i.e., compounds of the type RQ or R′Q, in which R and R′ are as defined above and Q denotes a leaving group such as a halide, a sulfate, etc.

Yet another preferred family of cationic associative polyurethanes is the one corresponding to formula (XXIV) above in which:

R and R′ both independently represent a hydrophobic group,
X and X′ both independently represent a group comprising a quaternary amine,
n and p are zero, and
L, L′, Y, and m have the meanings given above.

The number-average molecular mass of the cationic associative polyurethanes is preferably between 400 and 500 000, in particular between 1000 and 400 000, and ideally between 1000 and 300 000.

The expression “hydrophobic group” means a radical or polymer containing a saturated or unsaturated, linear or branched hydrocarbon-based chain, which may contain one or more heteroatoms such as P, O, N, or S, or a radical containing a perfluoro or silicone chain. When the hydrophobic group denotes a hydrocarbon-based radical, it comprises at least 10 carbon atoms, preferably from 10 to 30 carbon atoms, in particular from 12 to 30 carbon atoms, and more preferably from 18 to 30 carbon atoms.

Preferentially, the hydrocarbon-based group is derived from a monofunctional compound.

By way of example, the hydrophobic group may be derived from a fatty alcohol such as stearyl alcohol, dodecyl alcohol, or decyl alcohol. It may also denote a hydrocarbon-based polymer, for instance polybutadiene.

When X and/or X′ denote(s) a group comprising a tertiary or quaternary amine, X and/or X′ may represent one of the following formulae:

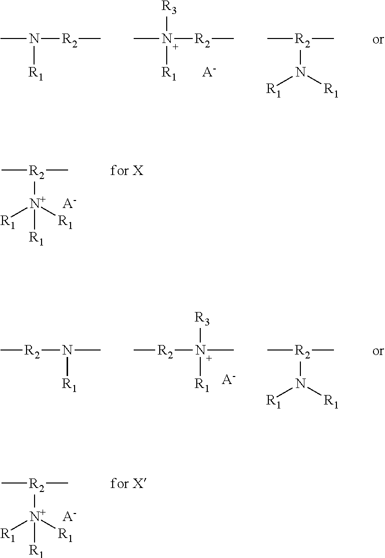

in which:
R₂ represents a linear or branched alkylene radical containing from 1 to 20 carbon atoms, optionally comprising a saturated or unsaturated ring, or an arylene radical, one or more of the carbon atoms possibly being replaced with a heteroatom chosen from N, S, O, and P;
R₁ and R₃, which may be identical or different, denote a C₁-C₃₀ alkyl or alkenyl radical or an aryl radical, at least one of the carbon atoms possibly being replaced with a heteroatom chosen from N, S, O, and P; and
A⁻ is a physiologically acceptable counterion.

The groups L, L′, and L″ represent a group of formula:

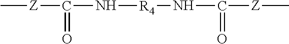

in which:
Z represents —O—, —S—, or —NH—; and
R₄ represents a linear or branched alkylene radical containing from 1 to 20 carbon atoms, optionally comprising a saturated or unsaturated ring, or an arylene radical, one or more of the carbon atoms possibly being replaced with a heteroatom chosen from N, S, O, and P.

The groups P and P′ comprising an amine function may represent at least one of the following formulae:

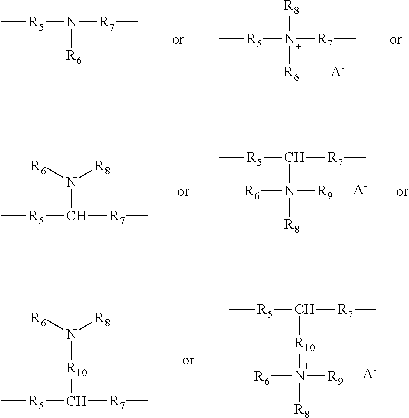

in which:
R₅ and R₇ have the same meaning as R₂ defined above;
R₆, R₈, and R₉ have the same meanings as R₁ and R₃ defined above;
R₁₀ represents a linear or branched, optionally unsaturated alkylene group possibly containing one or more heteroatoms chosen from N, O, S, and P; and
A⁻ is a physiologically acceptable counterion.

As regards the meaning of Y, the term “hydrophilic group” means a polymeric or non-polymeric water-soluble group.

By way of example, when it is not a polymer, mention may be made of ethylene glycol, diethylene glycol, and propylene glycol.

When it is a hydrophilic polymer, in accordance with one preferred embodiment, mention may be made, for example, of polyethers, sulfonated polyesters, sulfonated polyamides, or a mixture of these polymers. The hydrophilic compound is preferentially a polyether and especially a poly(ethylene oxide) or poly(propylene oxide).

The cationic associative polyurethanes of formula (XXIV) that may be used according to the present invention are formed from diisocyanates and from various compounds with functions containing a labile hydrogen. The functions containing a labile hydrogen may be alcohol, primary or secondary amine, or thiol functions, giving, after reaction with the diisocyanate functions, polyurethanes, polyureas, and polythioureas, respectively. The expression “polyurethanes” that may be used according to the present invention encompasses these three types of polymer, namely polyurethanes per se, polyureas, and polythioureas, and also copolymers thereof.

A first type of compound involved in the preparation of the polyurethane of formula (XXIV) is a compound comprising at least one unit containing an amine function. This compound may be multifunctional, but the compound is preferentially difunctional, that is to say that, according to one preferential embodiment, this compound comprises two labile hydrogen atoms borne, for example, by a hydroxyl, primary amine, secondary amine, or thiol function. A mixture of multifunctional and difunctional compounds in which the percentage of multifunctional compounds is low may also be used.

As mentioned above, this compound may comprise more than one unit containing an amine function. In this case, it is a polymer bearing a repetition of the unit containing an amine function.

Compounds of this type may be represented by one of the following formulae:

HZ—(P)n-ZH

Or

HZ—(P′)p-ZH

in which Z, P, P′, n, and p are as defined above.

Examples of compounds containing an amine function that may be mentioned include N-methyldiethanolamine, N-tert-butyldiethanolamine, and N-sulfoethyldiethanolamine.

The second compound included in the preparation of the polyurethane of formula (XVIII) is a diisocyanate corresponding to the formula:

O═C═N—R₄—N═C═O

in which R₄ is as defined above.

By way of example, mention may be made of methylenediphenyl diisocyanate, methylenecyclohexane diisocyanate, isophorone diisocyanate, tolylene diisocyanate, naphthalene diisocyanate, butane diisocyanate, and hexane diisocyanate.

A third compound involved in the preparation of the polyurethane of formula (XXIV) is a hydrophobic compound intended to form the terminal hydrophobic groups of the polymer of formula (XXIV).

This compound consists of a hydrophobic group and of a function containing a labile hydrogen, for example, a hydroxyl, primary or secondary amine, or thiol function

By way of example, this compound may be a fatty alcohol such as, in particular, stearyl alcohol, dodecyl alcohol, or decyl alcohol. When this compound comprises a polymeric chain, it may be, for example, α-hydroxylated hydrogenated polybutadiene.

The hydrophobic group of the polyurethane of formula (XXIV) may also result from the quaternization reaction of the tertiary amine of the compound comprising at least one tertiary amine unit. Thus, the hydrophobic group is introduced via the quaternizing agent. This quaternizing agent is a compound of the type RQ or R′Q, in which R and R′ are as defined above and Q denotes a leaving group such as a halide, a sulfate, etc.

The cationic associative polyurethane may also comprise a hydrophilic block. This block is provided by a fourth type of compound involved in the preparation of the polymer. This compound may be multifunctional. It is preferably difunctional. It is also possible to have a mixture in which the percentage of multifunctional compound is low.

The functions containing a labile hydrogen are alcohol, primary or secondary amine, or thiol functions. This compound may be a polymer terminated at the chain ends with one of these functions containing a labile hydrogen.

By way of example, when it is not a polymer, mention may be made of ethylene glycol, diethylene glycol, and propylene glycol.

When it is a hydrophilic polymer, mention may be made, for example, of polyethers, sulfonated polyesters, and sulfonated polyamides, or a mixture of these polymers. The hydrophilic compound is preferentially a polyether and especially a poly(ethylene oxide) or polypropylene oxide).

The hydrophilic group termed Y in formula (XXIV) is optional. Specifically, the units containing a quaternary amine or protonated function may suffice to provide the solubility or water-dispersibility required for this type of polymer in an aqueous solution.

Although the presence of a hydrophilic group Y is optional, cationic associative polyurethanes comprising such a group are, however, preferred.
(II) quaternized cellulose derivatives and polyacrylates containing non-cyclic amine side groups.

The quaternized cellulose derivatives are, in particular:
quaternized celluloses modified with groups comprising at least one fatty chain, such as alkyl, arylalkyl, or alkylaryl groups comprising at least 8 carbon atoms, or mixtures thereof,
quaternized hydroxyethylcelluloses modified with groups comprising at least one fatty chain, such as alkyl, arylalkyl, or alkylaryl groups comprising at least 8 carbon atoms, or mixtures thereof.

The alkyl radicals borne by the above quaternized celluloses or hydroxyethylcelluloses preferably contain from 8 to 30 carbon atoms. The aryl radicals preferably denote phenyl, benzyl, naphthyl, or anthryl groups.

Examples of quaternized alkylhydroxyethylcelluloses containing C₈-C₃₀ fatty chains that may be mentioned include the products Quatrisoft LM 200®, Quatrisoft LM-X 529-18-A®, Quatrisoft LM-X 529-18B® (C₁₂ alkyl), and Quatrisoft LM-X 529-8® (C₁₈ alkyl) sold by the company Amerchol, and the products Crodacel QM®, Crodacel QL® (C₁₂ alkyl), and Crodacel QS® (C₁₈ alkyl) sold by the company Croda.

The amphoteric associative polymers are preferably chosen from those comprising at least one non-cyclic cationic unit. Even more particularly, the ones that are preferred are those prepared from or comprising 1 to 20 mol %, preferably 1.5 to 15 mol %, and even more particularly 1.5 to 6 mol % of fatty-chain monomer relative to the total number of moles of monomers.

The amphoteric associative polymers that are preferred according to the present invention comprise or are prepared by copolymerizing:

1) at least one monomer of formula (XXV) or (XXVI):

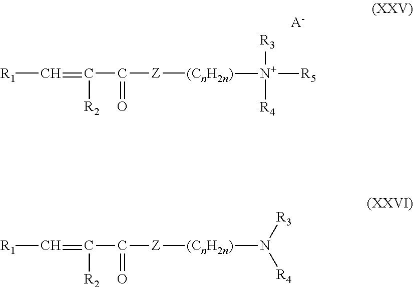

in which R₁ and R₂, which may be identical or different, represent a hydrogen atom or a methyl radical, R₃, R₄, and R₅, which may be identical or different, represent a linear or branched alkyl radical containing from 1 to 30 carbon atoms,
Z represents an NH group or an oxygen atom,
n is an integer from 2 to 5,
A⁻ is an anion derived from an organic or mineral acid, such as a methosulfate anion or a halide such as chloride or bromide;
2) at least one monomer of formula (XXVII):

R₆—CH═CR₇—COOH (XXVII)

in which R₆ and R₇, which may be identical or different, represent a hydrogen atom or a methyl radical;
and
3) at least one monomer of formula (XXVIII):

R₆—CH═CR₇—COXR₈ (XXVIII)

in which R₆ and R₇, which may be identical or different, represent a hydrogen atom or a methyl radical, X denotes an oxygen or nitrogen atom, and R₈ denotes a linear or branched alkyl radical containing from 1 to 30 carbon atoms;
at least one of the monomers of formula (XXV), (XXVI), or (XXVIII) comprising at least one fatty chain.

The monomers of formulae (XXV) and (XXVI) of the present invention are preferably chosen from the group consisting of:
dimethylaminoethyl methacrylate, dimethylaminoethyl acrylate,
diethylaminoethyl methacrylate, diethylaminoethyl acrylate,
dimethylaminopropyl methacrylate, dimethylaminopropyl acrylate, and
dimethylaminopropylmethacrylamide, dimethylaminopropylacrylamide, these monomers optionally being quaternized, for example, with a C₁-C₄ alkyl halide or a C₁-C₄ dialkyl sulfate.

More particularly, the monomer of formula (XXV) is chosen from acrylamidopropyltrimethyl-ammonium chloride and methacrylamidopropyltrimethylammonium chloride.

The monomers of formula (XXVII) of the present invention are preferably chosen from the group consisting of acrylic acid, methacrylic acid, crotonic acid, and 2-methylcrotonic acid. More particularly, the monomer of formula (XXI) is acrylic acid.

The monomers of formula (XXVIII) of the present invention are preferably chosen from the group formed from C₁₂-C₂₂ and more particularly C₁₆-C₁₈ alkyl acrylates or methacrylates.

The monomers constituting the fatty-chain amphoteric polymers of the present invention are preferably already neutralized and/or quaternized.

The ratio of the number of cationic charges/anionic charges is preferably equal to about 1.

The amphoteric associative polymers according to the present invention preferably comprise from 1 mol % to 10 mol % of the monomer comprising a fatty chain (monomer of formula (XXV), (XXVI), or (XXVIII)), and preferably from 1.5 mol % to 6 mol %.

The weight-average molecular weights of the amphoteric associative polymers according to the present invention may range from 500 to 50 000 000 and are preferably between 10 000 and 5 000 000.

The amphoteric associative polymers according to the present invention may also contain other monomers such as nonionic monomers and in particular such as C₁-C₄ alkyl acrylates or methacrylates.

Amphoteric associative polymers according to the present invention are described and prepared, for example, in patent application WO 98/44012.

Among the amphoteric associative polymers according to the present invention, the ones that are preferred are acrylic acid/(meth)acrylamidopropyltrimethylammonium chloride/stearyl methacrylate terpolymers.

The associative polymers of nonionic type that may be used according to the present invention are preferably chosen from:
(1) celluloses modified with groups comprising at least one fatty chain;
   examples that may be mentioned include:
hydroxyethylcelluloses modified with groups comprising at least one fatty chain, such as alkyl, arylalkyl, or alkylaryl groups, or mixtures thereof, and in which the alkyl groups are preferably C₈-C₂₂, for instance the product Natrosol Plus Grade 330 CS® (C₁₆ alkyl) sold by the company Aqualon, or the product Bermocoll EHM 100® sold by the company Berol Nobel,
hydroxyethylcelluloses modified with alkylphenyl polyalkylene glycol ether groups, such as the product Amercell Polymer HM-1500® (nonylphenyl polyethylene glycol (15) ether) sold by the company Amerchol.
(2) hydroxypropyl guars modified with groups comprising at least one fatty chain, such as the product Esaflor HM 22® (C₂₂ alkyl chain) sold by the company Lamberti, and the products RE210-180 (C₁₄ alkyl chain) and RE205-1® (C₂₀ alkyl chain) sold by the company Rhône-Poulenc.
(3) copolymers of vinylpyrrolidone and of fatty-chain hydrophobic monomers; examples that may be mentioned include:
the products Antaron V216® or Ganex V216® (vinylpyrrolidone/hexadecene copolymer) sold by the company ISP.
the products Antaron V220® or Ganex V220® (vinylpyrrolidone/eicosene copolymer) sold by the company ISP.
(4) copolymers of C₁-C₆ alkyl methacrylates or acrylates and of amphiphilic monomers comprising at least one fatty chain, for instance the oxyethylenated methyl acrylate/stearyl acrylate copolymer sold by the company Goldschmidt under the name Antil 208®.
(5) copolymers of hydrophilic methacrylates or acrylates and of hydrophobic monomers comprising at least one fatty chain, for instance the polyethylene glycol methacrylate/lauryl methacrylate copolymer.
(6) polyurethane polyethers comprising in their chain both hydrophilic blocks usually of polyoxyethylenated nature and hydrophobic blocks, which may be aliphatic sequences alone and/or cycloaliphatic and/or aromatic sequences.
(7) polymers with an aminoplast ether backbone containing at least one fatty chain, such as the Pure Thix® compounds sold by the company Sud-Chemie.

Preferably, the polyurethane polyethers comprise at least two hydrocarbon-based lipophilic chains containing from 6 to 30 carbon atoms, separated by a hydrophilic block, the hydrocarbon-based chains possibly being pendent chains or chains at the end of the hydrophilic block. In particular, it is possible for one or more pendent chains to be included. In addition, the polymer may comprise a hydrocarbon-based chain at one end or at both ends of a hydrophilic block.

The polyurethane polyethers may be multiblock, in particular in triblock form. The hydrophobic blocks may be at each end of the chain (for example: triblock copolymer containing a hydrophilic central block) or distributed both at the ends and in the chain (for example, multiblock copolymer). These same polymers may also be graft polymers or star polymers.

The nonionic fatty-chain polyurethane polyethers may be triblock copolymers in which the hydrophilic block is a polyoxyethylenated chain comprising from 50 to 1000 oxyethylene groups. The nonionic polyurethane polyethers comprise a urethane linkage between the hydrophilic blocks, whence arises the name.

By extension, also included among the nonionic fatty-chain polyurethane polyethers are those in which the hydrophilic blocks are linked to the lipophilic blocks via other chemical bonds.

As examples of nonionic fatty-chain polyurethane polyethers that may be used in the present invention, it is also possible to use Rheolate 205® containing a urea function, sold by the company Rheox, or Rheolate® 208, 204, or 212, and also Acrysol RM 184®.

Mention may also be made of the product Elfacos T210@ containing a C₁₂-C₁₄ alkyl chain, and the product Elfacos T212® containing a C₁₈ alkyl chain, from Akzo.

The product DW 1206B® from Rohm & Haas containing a C₂₀ alkyl chain and a urethane bond, sold at a solids content of 20% in water, may also be used.

It is also possible to use solutions or dispersions of these polymers, especially in water or in aqueous-alcoholic medium. Examples of such polymers that may be mentioned are Rheolate® 255, Rheolate® 278, and Rheolate® 244 sold by the company Rheox. The products DW 1206F and DW 1206J sold by the company Rohm & Haas may also be used.

The polyurethane polyethers that may be used according to the present invention are in particular those described in the article by G. Fonnum, J. Bakke, and Fk. Hansen-Colloid Polym. Sci 271, 380.389 (1993).

It is even more particularly preferred to use a polyurethane polyether that may be obtained by polycondensation of at least three compounds comprising (i) at least one polyethylene glycol comprising from 150 to 180 mol of ethylene oxide, (ii) stearyl alcohol or decyl alcohol, and (iii) at least one diisocyanate.

Such polyurethane polyethers are sold especially by the company Rohm & Haas under the names Aculyn 46® and Aculyn 44® [Aculyn 46® is a polycondensate of polyethylene glycol containing 150 or 180 mol of ethylene oxide, of stearyl alcohol, and of methylenebis(4-cyclohexyl isocyanate) (SMDI), at 15% by weight in a matrix of maltodextrin (4%) and water (81%); Aculyn 44® is a polycondensate of polyethylene glycol containing 150 or 180 mol of ethylene oxide, of decyl alcohol, and of methylenebis(4-cyclohexyl isocyanate) (SMDI), at 35% by weight in a mixture of propylene glycol (39%) and water (26%)].

Among the said above associative polymers, those most preferred according to the present invention are anionic associative polymers, in particular acrylate/C₁₀₋₃₀ alkyl acrylate copolymers (INCI name: Acrylates/C10-30 Alkyl Acrylate Crosspolymer), such as Pemulen TR1®, Pemulen TR2®, Carbopol 1382®, and Carbopol EDT2020@ from Lubrizol, and TEGO® Carbomer 841 SER from Evonik, even more preferentially TEGO® Carbomer 841 SER.

The amount of the (g) associative polymer is not limited, and may range from 0.01 to 10% by weight, preferably from 0.05 to 5% by weight, and more preferably from 0.1 to 2% by weight, relative to the total weight of the composition.

### [Cosmetic Active Ingredients]

The composition according to the present invention may further comprise at least one cosmetic active ingredient. A single type of cosmetic active ingredient may be used, but two or more different types of cosmetic active ingredient may be used in combination.

It is preferable that the (h) cosmetic active ingredient be selected from the group consisting of whitening agents, anti-aging agents, UV filters, keratolytic and anti-bacterial agents.

It is preferable that the (h) cosmetic active ingredient be selected from the group consisting of oxothiazolidinecarboxylic acid, Vitamin B3 and derivatives thereof, preferably niacinamide, Vitamin C and derivatives thereof, preferably 3-O-ethyl ascorbic acid, resorcinol and derivatives thereof such as phenylethyl resorcinol, xanthine bases, preferably caffeine, camphor benzalkonium methosulfate, ellagic acid, hydroxyphenoxy propionic acid, diethyllutidinate, terephthalylidene dicamphor sulfonic acid, ferulic acid, salicylic acid, phloretine, acetyl trifluoromethylphenyl valylglycine, resveratrol, apigenin, prasterone, benzophenone-3, butyl methoxydibenzoylmethane, capryloyl salicylic acid, ethylhexyl salicylate, and jasmonic acid derivatives, preferably sodium tetrahydrojasmonate.

The (h) cosmetic active ingredient may be present in an amount ranging from 0.001% to 10% by weight, and preferably from 0.01% to 5% by weight, such as from 0.01% to 1% by weight, relative to the total weight of the composition.

### [Polyol]

The composition according to the present invention may further comprise at least one polyol. A single type of polyol may be used, but two or more different types of polyol may be used in combination.

The term “polyol” here means an alcohol having two or more hydroxy groups, and does not encompass a saccharide or a derivative thereof. The derivative of a saccharide includes a sugar alcohol which is obtained by reducing one or more carbonyl groups of a saccharide, as well as a saccharide or a sugar alcohol in which the hydrogen atom or atoms in one or more hydroxy groups thereof has or have been replaced with at least one substituent such as an alkyl group, a hydroxyalkyl group, an alkoxy group, an acylgroup or a carbonyl group.

The polyol may be a C₂-C₁₂ polyol, preferably a C₂-C₉ polyol, comprising at least 2 hydroxy groups, and preferably 2 to 5 hydroxy groups.

The polyol may be a natural or synthetic polyol. The polyol may have a linear, branched or cyclic molecular structure.

The polyol may be selected from glycerins and derivatives thereof, and glycols and derivatives thereof. The polyol may be selected from the group consisting of glycerin, diglycerin, polyglycerin, ethyleneglycol, diethyleneglycol, propyleneglycol, dipropyleneglycol, butyleneglycol, pentyleneglycol, hexyleneglycol, 1,3-propanediol, 1,5-pentanediol, polyethyleneglycol (5 to 50 ethyleneoxide groups), and sugars such as sorbitol.

The polyol may be present in an amount ranging from 0.01% to 30% by weight, and preferably from 0.1% to 30% by weight, such as from 1% to 25% by weight, relative to the total weight of the composition.

### [Other Ingredients]

The composition according to the present invention may also comprise an effective amount of other ingredients, known previously elsewhere in lightening or coloring compositions, such as various common adjuvants, sequestering agents such as EDTA and etidronic acid, preserving agents, vitamins or provitamins different from those mentioned above, for instance, panthenol, opacifiers, fragrances, plant extracts, cationic polymers and so on.

The composition according to the present invention may further comprise at least one organic solvent. So the organic solvent is preferably water miscible. As the organic solvent, there may be mentioned, for example, C₁-C₄ alkanols, such as ethanol and isopropanol; aromatic alcohols such as benzyl alcohol and phenoxyethanol; analogous products; and mixtures thereof.

The organic water-soluble solvents may be present in an amount ranging from less than 10% by weight, preferably from 5% by weight or less, and more preferably from 1% by weight or less, relative to the total weight of the composition.

[Preparation and Properties]

The composition according to the present invention can be prepared by mixing the above essential and optional ingredients in accordance with a conventional process. The conventional process includes mixing with a homogenizer, for example a turbine mixer.

It may be preferable that the weight ratio of the (b) polyglyceryl fatty acid ester to the (a) oil may be 1 or less, preferably 0.8 or less, and more preferably 0.6 or less. The weight ratio of the (b) polyglyceryl fatty acid ester/the (a) oil is preferably 0.01 or more, preferably from 0.05 or more.

It may also be preferable that the weight ratio of the (b) polyglyceryl fatty acid ester to the (c) silicone elastomer may be 15 or less, preferably 12 or less, and more preferably 10 or less. If this weight ratio is more than 15, the composition according to the present invention may tend to show stickiness.

It is preferable that the composition according to the present invention be in the form of an O/W (oil-in-water) type emulsion in which an oily phase containing liquid oils and silicones is dispersed in water phase.

It is more preferable that the composition according to the present invention be in the form of Si/Wm (silicone-in-O/W type micro-emulsion) type emulsion in which silicone is dispersed in O/W (oil-in-water) type micro-emulsion (Wm) in which oil is solubilized by micelles.

The “micro-emulsion” may be defined in two ways, namely, in a broad sense and in a narrow sense. That is to say, there are the one case (“micro-emulsion in the narrow sense”) in which the micro-emulsion refers to a thermodynamically stable isotropic single liquid phase containing a ternary system having three ingredients of an oily component, an aqueous component and a surfactant, and the other case (“micro-emulsion in the broad sense”) in which among thermodynamically unstable typical emulsion systems the micro-emulsion additionally includes those such emulsions presenting transparent or translucent appearances due to their smaller particle sizes (Satoshi Tomomasa, et al., Oil Chemistry, Vol. 37, No. 11 (1988), pp. 48-53). The “micro-emulsion” as used herein refers to a “micro-emulsion in the narrow sense”, i.e., a thermodynamically stable isotropic single liquid phase.

The micro-emulsion refers to either one state of an O/W (oil-in-water) type micro-emulsion in which oil is solubilized by micelles, a W/O (water-in-oil) type micro-emulsion in which water is solubilized by reverse micelles, or a bicontinuous micro-emulsion in which the number of associations of surfactant molecules are rendered infinite so that both the aqueous phase and oil phase have a continuous structure.

The micro-emulsion may have a dispersed phase with a number average diameter of 100 nm or less, preferably 50 nm or less, and more preferably 20 nm or less, measured by laser granulometry.

The micro-emulsion may have a dispersed phase with a number average diameter of 300 nm or less, preferably 200 nm or less, and more preferably 100 nm or less, measured by laser granulometry.

In the O/W emulsion of the composition according to the present invention, oily phases dispersed in a continuous aqueous phase contain oils.

If the (a) oil includes a substantial amount of silicone oil, the composition according to the present invention may be in the form of a Si/W (silicone-in-water) type emulsion, preferably a Si/Wm emulsion (silicone-in-O/W type micro-emulsion) in which silicone is dispersed in O/W (oil-in-water) type micro-emulsion (Wm) in which oil is solubilized by micelles.

### [Process and Use]

It is preferable that the composition according to the present invention be a cosmetic composition, preferably a cosmetic composition for a keratin substance such as skin.

The composition according to the present invention can be used for a non-therapeutic process, such as a cosmetic process, for treating the skin, the hair, mucous membranes, the nails, the eyelashes, the eyebrows and/or the scalp, by being applied to the skin, the hair, mucous membranes, the nails, the eyelashes, the eyebrows or the scalp.

The present invention may also relate to a use of the composition according to the present invention as a cosmetic product or in a cosmetic product such as care products, washing products, make-up products, make-up-removing products, for body and/or facial skin and/or mucous membranes and/or the scalp and/or the hair and/or the nails and/or the eyelashes and/or the eyebrows.

In other words, the composition according to the present invention can be used, as it is, as a cosmetic product. Alternatively, the composition according to the present invention can be used as an element of a cosmetic product. For example the composition according to the present invention can be added to or combined with any other elements to form a cosmetic product.

The care product may be a lotion, a cream, a hair tonic, a hair conditioner, a sun screening agent, and the like. The washing product may be a shampoo, a face wash, a hand wash and the like. The make-up product may be a foundation, a mascara, a lipstick, a lip gloss, a blusher, an eye shadow, a nail varnish, and the like. The make-up-removing product may be a make-up cleansing agent and the like.

### Examples

The present invention will be described in more detail by way of examples, which however should not be construed as limiting the scope of the present invention.

### Examples 1-5 and Comparative Examples 1-4

The following compositions according to Examples 1-5 and Comparative Examples 1-4, shown in

Table 1, were prepared by mixing the components shown in Table 1 as follows: (1) mixing the ingredients of phase A at 75-85° C. to form an oil phase; (2) mixing the ingredients of phase C at 75-85° C. to form an aqueous phase; (3) adding the aqueous phase to the oil phase, followed by mixing and maintaining the obtained mixture at 60-70° C.; (4) mixing the ingredients of phase B at room temperature; and (5) adding the mixture of the ingredients of phase B to the mixture of phases A and C at 60-70° C., followed by homogenizing them to obtain an O/W emulsion, more specifically Si/Wm emulsion. The obtained emulsion was cooled to 30° C. or less. The numerical values for the amounts of the components shown in Table 1 are all based on “% by weight” as active raw materials.

TABLE 1

| | | | | | | | Comp. | Comp. | Comp. | Comp. |
|---|---|---|---|---|---|---|---|---|---|---|
| Phase | Ingredients | Ex. 1 | Ex. 2 | Ex. 3 | Ex. 4 | Ex. 5 | Ex. 1 | Ex. 2 | Ex. 3 | Ex. 4 |
| A | (a) Octyldodecanol | — | — | — | 2 | 2 | — | — | — | — |
| | (a) Ethylhexyl Palmitate | 3 | 3 | — | — | — | — | 3 | 3 | 3 |
| | (a) Dicaprylyl Carbonate | — | — | 3 | — | — | — | — | — | — |
| | (a) Hexyldecanol | — | — | — | 1 | 1 | — | — | — | — |
| | (b) Polyglyceryl-5 Laurate | 3 | 3 | 3 | 3 | 3.5 | 3 | — | 3 | 3 |
| | (b) Polyglyceryl-2 Laurate | — | — | — | — | 0.5 | — | — | — | — |
| B | (c) Dimethicone/Vinyl Dimethicone | 0.36 | 2.4 | 1.44 | 1.44 | 1.44 | 0.36 | 0.36 | — | 0.36 |
| | Crosspolymer | | | | | | | | | |
| | (a) Dimethicone | 1.14 | 7.6 | 4.56 | 4.56 | 4.56 | — | 1.14 | — | 1.14 |
| | (c) Polysilicone-11 | — | — | 1.95 | 1.95 | 1.95 | — | — | — | — |
| | (a) Cyclohexasiloxane | — | — | 13.05 | 13.05 | 13.05 | — | — | — | — |
| | (a) Dimethicone (and) Dimethiconol | 1.5 | 1.5 | 2 | 2 | 2 | — | 1.5 | 1.5 | 1.5 |
| | (d) Sclerotium Gum | 0.5 | 0.5 | 0.3 | 0.3 | 0.3 | 0.5 | 0.5 | 0.5 | — |
| | (d) Xanthan Gum | — | — | — | 0.25 | 0.2 | — | — | — | — |
| | (g) Acrylates/C10-30 Alkyl Acrylate | 0.35 | 0.35 | 0.35 | — | — | 0.35 | 0.35 | 0.35 | 0.35 |
| | Crosspolymer | | | | | | | | | |
| C | (f) PPG-6 Decyltetradeceth-30 | — | — | 0.2 | 1 | 1 | — | — | — | — |
| | (f) Sodium Methyl Stearoyl Taurate | 0.2 | 0.2 | 0.35 | 0.2 | 0.2 | 0.2 | 0.2 | 0.2 | 0.2 |
| | (f) Sodium Stearoyl Glutamate | — | — | — | 0.2 | — | — | — | — | — |
| A | (h) Phenylethyl Resorcinol | — | — | 0.3 | 0.3 | 0.3 | — | — | — | — |
| C | (h) 3-O-Ethyl Ascorbic Acid | 1 | 1 | — | — | — | 1 | 1 | 1 | 1 |
| | (h) Niacinamide | 1 | 1 | — | — | — | 1 | 1 | 1 | 1 |
| | Butyleneglycol | 2.5 | 2.5 | 2.5 | 2.5 | 5 | 2.5 | 2.5 | 2.5 | 2.5 |
| | Glycerin | 3 | 3 | 3 | 3 | 3 | 3 | 3 | 3 | 3 |
| | Disodium EDTA | 0.1 | 0.1 | 0.1 | 0.1 | 0.1 | 0.1 | 0.1 | 0.1 | 0.1 |
| | Sodium Citrate | 0.284 | 0.284 | — | — | — | 0.284 | 0.284 | 0.284 | 0.284 |
| | Citric Acid | 0.146 | 0.146 | — | — | — | 0.146 | 0.146 | 0.146 | 0.146 |
| | Salicylic Acid | 0.2 | 0.2 | — | — | — | 0.2 | 0.2 | 0.2 | 0.2 |
| | Capryloyl Salicylic Acid | — | — | 0.3 | 0.3 | 0.3 | — | — | — | — |
| | Sodium Hydroxide | — | — | 0.074 | 0.074 | 0.074 | — | — | — | — |
| | Preservative | 0.6 | 0.6 | 0.6 | 0.6 | 0.6 | 0.6 | 0.6 | 0.6 | 0.6 |
| | (e) Water | qs 100 | qs 100 | qs 100 | qs 100 | qs 100 | qs 100 | qs 100 | qs 100 | qs 100 |

| Non-Sticky Feeling After Application | | Good | Very | Very | Very | Very | Good | Good | Poor | Good |
|---|---|---|---|---|---|---|---|---|---|---|
| | | | Good | Good | Good | Good | | | | |
| Stability | Immediately After Preparation | Very | Good | Good | Good | Good | Very | Very | Good | Good |
| | | Good | | | | | Poor | Poor | | |
| | 2 Months Later at 45° C. | Very | Good | Good | Good | Good | Very | Very | Good | Poor |
| | | Good | | | | | Poor | Poor | | |

Dimethicone/Vinyl Dimethicone Crosspolymer and Dimethicone: KSG-16 (Shin-Etsu), Dimethicone/Vinyl Dimethicone Crosspolymer:Dimethicone = 24:76 (weight ratio)
Dimethicone/Vinyl Dimethicone Crosspolymer: TREFIL ® E-506 S (Dow Corning Toray)
Polysilicone-11 and Cyclohexasiloxane: Gransil RPS-D6 (Grant Industries), Polysilicone-11:Cyclohexasiloxane = 13:87 (weight ratio)
Dimethicone (and) Dimethiconol: Xiameter PMX-1503 Fluid (Dow Corning)

### [Evaluation]

### (Non-Sticky Feeling After Application)

5 professional panelists evaluated “non-sticky feeling after application” for the compositions according to Examples 1-5 and Comparative Examples 1-4. Each panelist took each composition in their hands, then applied it onto their faces to evaluate non-sticky feeling after application, and graded it from 1 (poor) to 5 (very good), which was then classified in the following 4 categories based on the average of the grade:

### Very Good: From 5.0 to 4.0

### Good: From 3.9 to 3.0

### Poor: From 2.9 to 2.0

### Very Poor: From 1.9 to 1.0

The results are shown in Table 1.

### (Stability)

### (1) Immediately After Preparation

Each of the compositions according to Examples 1-5 and Comparative Examples 1-4 was evaluated immediately after the preparation of each composition, in terms of the emulsion state by visual and microscopic observation, and was evaluated in accordance with the following criteria:

Very Good: No separation. Very fine emulsion was observed.
Good: No separation. Fine emulsion was observed.
Poor: Separation. Coarse emulsion was clearly observed.
Very Poor: Separation. Very coarse emulsion was remarkably noticed.

The results are shown in Table 1.

### (2) 2 Months Later at 45° C.

Each of the compositions according to Examples 1-5 and Comparative Examples 1-4 was filled into a glass bottle and was held under temperature conditions of 45° C. for 2 months. Each sample was then investigated for the degree of change (color, odor, pH, viscosity, and emulsion state), and evaluated in accordance with the following criteria:

Very Good: Almost the same condition as production.
Good: Changes in color, odor, pH, viscosity, and emulsion state were somewhat observed. However, no separation was observed. A fine emulsion was observed and maintained.
Poor: Changes in color, odor, pH, viscosity, and emulsion state were clearly observed. Separation was clearly observed. Also, a coarse emulsion was clearly observed.
Very Poor: Changes in color, odor, pH, viscosity, and emulsion state were remarkably noticed. Separation was also remarkably noticed. Also, a coarse emulsion was remarkably observed.

As is clear from the results shown in Table 1, it was found that the composition in the form of an O/W emulsion according to the present invention (Examples 1-5) can provide both a non-sticky feeling after application and stability immediately after preparation and 2 months later at higher temperature, due to the combination of the oil (octyldodecanol, ethylhexylpalmitate, dicaprylyl carbonate or hexyldecanol), polyglyceryl fatty acid ester (at least polyglyceryl-5 laurate), organopolysiloxane elastomer (dimethicone/vinyldimethicone crosspolymer), and polysaccharide (at least sclerotium gum). Thus, the composition according to the present invention can provide an excellent feeling to the touch and stability even after a long time period.

On the other hand, Comparative Example 1 which lacks oil ingredients, cannot show excellent stability. Also, Comparative Example 2, which lacks polyglyceryl fatty acid ester, cannot show excellent stability. Comparative Example 3, which lacks organopolysiloxane elastomer, cannot show an excellent feeling to the touch. Comparative Example 4, which lacks polysaccharide, cannot show stability over time.

1. A composition comprising:
(a) at least one oil;
(b) at least one polyglyceryl fatty acid ester;
(c) at least one silicone elastomer; and
(d) at least one polysaccharide.

2. The composition according to claim 1, wherein the (a) oil comprises at least one ester oil, at least one fatty alcohol or a mixture thereof.

3. The composition according to claim 1 or 2, wherein the amount of the (a) oil ranges from 0.1 to 35% by weight, preferably from 0.5 to 30% by weight, and more preferably from 1 to 25% by weight, relative to the total weight of the composition.

4. The composition according to any one of claims 1 to 3, wherein the (b) polyglyceryl fatty acid ester has a polyglyceryl moiety derived from 2 to 10 glycerins, preferably 2 to 8 glycerins, and more preferably from 2 to 6 glycerins.

5. The composition according to any one of claims 1 to 4, wherein the (b) polyglyceryl fatty acid ester is chosen from polyglyceryl monolaurate comprising 2 to 6 glycerol units, polyglyceryl mono(iso)stearate comprising 2 to 6 glycerol units, polyglyceryl monooleate comprising 2 to 6 glycerol units, and polyglyceryl dioleate comprising 2 to 6 glycerol units.

6. The composition according to any one of claims 1 to 5, wherein the amount of the (b) polyglyceryl fatty acid ester ranges from 0.1 to 20% by weight, preferably from 0.5 to 15% by weight, and more preferably from 1 to 10% by weight, relative to the total weight of the composition.

7. The composition according to any one of claims 1 to 6, wherein the weight ratio of the (b) polyglyceryl fatty acid ester to the (a) oil is 1 or less, preferably 0.8 or less, and more preferably 0.6 or less.

8. The composition according to any one of claims 1 to 7, wherein the weight ratio of the (b) polyglyceryl fatty acid ester to the (c) silicone elastomer is 15 or less, preferably 12 or less, and more preferably 10 or less.

9. The composition according to any one of claims 1 to 8, wherein the amount of the (c) silicone elastomer ranges from 0.05 to 15% by weight, preferably from 0.1 to 10% by weight, and more preferably from 0.2 to 5% by weight, relative to the total weight of the composition.

10. The composition according to any one of claims 1 to 9, wherein the (d) polysaccharide is derived from microorganisms.

11. The composition according to any one of claims 1 to 10, wherein the amount of the (d) polysaccharide ranges from 0.01 to 10% by weight, preferably from 0.05 to 5% by weight, and more preferably from 0.1 to 1% by weight, relative to the total weight of the composition.

12. The composition according to any one of claims 1 to 11, wherein the composition further comprises water.

13. The composition according to any one of claims 1 to 12, wherein the composition further comprises at least one additional surfactant different from the (b) polyglyceryl fatty acid ester.

14. The composition according to any one of claims 1 to 13, wherein the composition further comprises at least one associative polymer.

15. A cosmetic process for treating the skin, the hair, mucous membranes, the nails, the eyelashes, the eyebrows and/or the scalp, characterized in that the composition according to any one of claims 1 to 14 is applied to the skin, the hair, mucous membranes, the nails, the eyelashes, the eyebrows or the scalp.
